(19) United States
(12) Patent Application Publication
(10) Pub. No.: US 2026/0297103 A1
(43) Pub. Date: Oct. 1, 2026

(54) PROCESS FOR PREPARING BENZGALANTAMINE AND SALTS THEREOF

(71) Applicant: Alpha Cognition Inc., Vancouver (CA)

(72) Inventors: Denis G. Kay, Stanhope (CA); Fred D. Sancilio, Stuart, FL (US); Haranath Kumar Vaddi, Weston, FL (US)

(21) Appl. No.: 19/090,242

(22) Filed: Mar. 25, 2025

(51) Int. Cl. C07D 491/06; A61K 31/55

(52) U.S. Cl. CPC C07D 491/06; A61K 31/55

(57) ABSTRACT

A process for preparing benzgalantamine comprising the addition of a benzoyl halide to galantamine. The process comprising additionally the formation of a salt of benzgalantamine subsequent to the preparation of benzgalantamine. The disclosure further relates to a composition comprising or consisting of a reaction product prepared by the process according to the present invention. The disclosure further relates to the composition comprising one or more specific impurities, preferably at negligible amounts. The disclosure further relates to a pharmaceutical composition comprising the composition together with a pharmaceutically acceptable carrier.

The invention is in the field of chemistry and pharmaceuticals, in particular in the field of organic synthesis of pharmaceutical compounds, more particularly regarding the synthesis of benzgalantamine (galantamine benzoate) and salts thereof.

The invention relates to a process for preparing benzgalantamine comprising an esterification step of galantamine by a benzoate. The invention further relates to a process for preparing benzgalantamine comprising the addition of a benzoyl halide to a galantamine salt. The invention further relates to the process comprising additionally the formation of a salt of benzgalantamine subsequent to the preparation of benzgalantamine.

The invention further relates to a composition comprising or consisting of a reaction product prepared by the process according to the present invention. The invention further relates to the composition comprising one or more specific impurities, preferably at negligible amounts. The invention further relates to a pharmaceutical composition comprising the composition according to the present invention together with a pharmaceutically acceptable carrier.

### BACKGROUND OF THE INVENTION

Galantamine, chemically known as (4aS,6R,8aS)-3-Methoxy-11-methyl-4a,5,9,10,11,12-hexahydro-6H-benzofuro[3a,3,2-ef][2]benzazepin-6-ol (CAS 357-70-0), is a tertiary amide, belonging to the phenanthrene chemical class, which occurs naturally in bulb plants and can also be produced synthetically. Galantamine is an inhibitor of the acetylcholinesterase (AChE) and further enhances cholinergic activity by non-competitive, allosteric modulation of the nicotinic acetylcholine receptors (nAChR), in particular the α7 subtype of nAChR. This slows the degradation of acetylcholine (ACh) and increases responsiveness of nAChR receptors to ACh. This mechanism of action results in an improvement of the cholinergic transmission, which is impaired in Alzheimer's disease.

Galantamine was introduced as a drug for Alzheimer's disease in 2000 and is approved in several countries worldwide. The approved indication is generally mild to moderate dementia of Alzheimer's type. It is currently available as an immediate release tablet, oral solution and extended-release capsule and tablet. It is marketed as RAZADYNE® ER in the U.S., and as REMINYL® elsewhere. Several generic equivalents have been approved by the Food and Drug Administration (FDA) and in several European countries by the European Medicines Agency (EMA).

In contrast to rivastigmine and donepezil, galantamine however does not significantly enrich in the human brain in comparison to blood plasma. This is because galantamine, a plant alkaloid, is much less lipophilic than the other two cholinesterase inhibitors used as drugs in AD and hence exhibits in steady-state only a rather low brain-to-blood concentration ratio (BBCR <2).

Through their primary action, cholinomimetics, such as galantamine, further increase gastric acid secretion due to increased cholinergic activity. Like other cholinesterase inhibitors, the most common adverse effects of galantamine (≥5% of adverse effects), in particular when applied orally, occur within the gastrointestinal tract (GIT), including nausea, vomiting, diarrhea, dizziness, headache, and decreased appetite. The most common adverse reactions associated with discontinuation of therapy in galantamine-treated patients from double-blind clinical trials were nausea, vomiting, decreased appetite, and dizziness.

During oral therapy with galantamine, the initial dose administered is therefore low and then subsequently increased over a period of months, adjusted according to the level of side effects perceived as acceptable by the patient. As a consequence of the side effects, many patients thus never reach a therapeutically most effective dose or discontinue therapy altogether. The majority of adverse reactions related to the gastrointestinal tract occurred during the dose-escalation period. In those patients who experienced the most frequent adverse reaction, nausea, the median duration of the nausea was 5-7 days.

Galantamine has been chemically modified to improve its lipophilicity and thus its passage through the blood-to-brain barrier (BBB) and mucosal tissue. Galantamine derivatives and prodrugs are described in EP 1940817 B1, WO 2009/127218 A1 and US 2009/0253654 A1.

The galantamine prodrug benzgalantamine, also termed galantamine benzoate, or ALPHA-1062, chemically known as ((4aS,6R,8aS)-4a,5,9,10,11,12-hexahydro-3-methoxy-11-methyl-6H-benzofuro[3a,3,2-ef][2]benzazepin-6-benzoate), is a benzoic ester of galantamine that exhibits limited or no pharmacological activity at therapeutic doses, until it is cleaved by esterase activity or in acidic conditions, which results in release of galantamine. There is substantial evidence from animal studies that intravenous, intranasal, buccal or sublingual administration of benzgalantamine rapidly achieves higher brain concentrations of benzgalantamine and galantamine than intravenous or oral administration of galantamine, and with a proportionally higher brain:blood concentration ratio. Benzgalantamine enhances delivery of galantamine to the brain, reduces GI side effects and therefore offers advantages over other drugs currently available for AD.

Means for the production of benzgalantamine and salts thereof are disclosed in WO 2009/127218 and WO2014/016430. WO 2009/127218 discloses a different procedure for the production of benzgalantamine. WO2014/016430 discloses methods of producing different salts of benzgalantamine.

However, the synthesis procedures disclosed in the prior art are not optimized for yield, and provide poor control of impurities, and of specific crystal forms of benzgalantamine salts, produced during the process.

Despite synthesis procedures known in the prior art, there is therefore a need for improved or alternative means for the synthesis of benzgalantamine and salts thereof as promising prodrugs for the treatment of several neurological diseases, such as dementia of Alzheimer's type. In particular, there is the need for simple, straight-forward, easy to control and scalable processes for the synthesis of benzgalantamine and salts thereof that allow the production of benzgalantamine at high yield and high purity.

### SUMMARY OF THE INVENTION

In light of the prior art, the technical problem underlying the present invention is to provide improved and/or alternative means for the preparation of benzgalantamine and salts thereof that overcome the disadvantages of the prior art.

Another problem underlying the present invention is to provide improved and/or alternative means for the preparation of benzgalantamine and salts thereof that are easy to control and scalable, allowing the production of benzgalantamine and salt thereof at high yields, high amounts and high purity.

Another problem underlying the present invention is to provide improved and/or alternative means for the preparation of benzgalantamine and salts thereof that are time- and cost-efficient.

Another problem underlying the present invention is to provide improved and/or alternative means for the preparation of benzgalantamine and salts thereof that allow the production of enantiomerically pure (or essentially pure) benzgalantamine or salts thereof.

Another problem underlying the present invention is to provide improved and/or alternative means for the preparation of benzgalantamine and salts thereof that allow control of the crystalline form of the salt product. Another problem underlying the present invention is to provide improved and/or alternative means for the preparation of benzgalantamine and salts thereof that allow the production of benzgalantamine gluconate as Form A.

These problems are solved by the features of the independent claims. Preferred embodiments of the present invention are provided in the dependent claims.

In one aspect, the invention relates to a process for preparing benzgalantamine

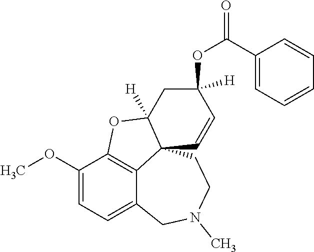 [SMILES: COc1ccc2c3c1O[C@H]1C[C@@H](OC(=O)c4ccccc4)C=C[C@@]31CCN(C)C2]

comprising addition of a benzoyl halide to a galantamine salt.

Advantageously, the process of the present invention offers several advantages and/or benefits that enhance its industrial applicability, and the synthesis product quality, compared to the prior art.

The process disclosed herein allows the production of benzgalantamine as a prodrug of galantamine at high reproducibility and high yields and further provides precise control over starting material and process-related impurities, allowing the production of a compound with consistently high purity and reproducible characteristics.

Surprisingly the process further allows for precise control of the crystalline form of the product, preferably Form A of benzgalantamine gluconate, which is important with regards to the product's physicochemical properties, such as solubility and stability, which also impact its bioavailability in therapeutic applications. Thereby, advantageously, no additional recrystallization step is required to obtain Form A.

In addition, the method advantageously allows to control enantiomeric purity. As shown in the examples below, the amount of “epi-benzgalantamine” related impurities and “(−)-benzgalantamine” can be controlled and limited. According to the method of the present invention, advantageously, benzgalantamine can be produced in an enantiomerically and stereoisomerically pure form (i.e., (+)-benzgalantamine).

Further, the scalability of the process advantageously allows it to be adapted from a laboratory to a commercial scale without compromising yield, purity, or reproducibility. This advantageously makes the process suitable for large-scale manufacturing, reducing the need for extensive reformulation or process modifications.

In embodiments, the invention relates to a process for preparing benzgalantamine, comprising addition of a benzoyl halide to galantamine (any given preparation of galantamine). All embodiments regarding the method may relate to the starting material of a galantamine salt or any other form or preparation of galantamine, to which a benzoyl halide is added.

In one embodiment of the invention, the addition takes place in an organic solvent in the presence of a catalyst, preferably a nucleophilic catalyst, and a base.

In one embodiment, the benzoyl halide is benzoyl chloride.

In one embodiment, the galantamine salt is galantamine hydrobromide.

In one embodiment, the galantamine salt is dissolved in an organic solvent. In one embodiment, galantamine hydrobromide is dissolved in an organic solvent. In one embodiment, a galantamine salt is dissolved in acetonitrile (ACN). In one embodiment, galantamine hydrobromide is dissolved in acetonitrile (ACN).

In one embodiment, a base is added to the solution of the galantamine salt in the organic solvent.

In one embodiment, the base is added to the solution of the galantamine salt in the organic solvent at ≤5 equivalents of the galantamine salt, preferably ≤4.5 equivalents, more preferably at 4 equivalents. In one embodiment, the base is added to the solution of the galantamine salt in the organic solvent at ≤5 equivalents of the galantamine salt, such as 5, 4.9, 4.8, 4.7, 4.6, 4.5, 4.4, 4.3, 4.2, 4.1, 4, 3.9, 3.8, 3.7, 3.6, 3.5, 3.4, 3.3, 3.2, 3.1 or 3 equivalents.

In one embodiment, the base is triethylamine (Et₃N) and is added to the solution of the galantamine salt in the organic solvent. In one embodiment, Et₃N is added to the solution of the galantamine salt in the organic solvent at ≤5 equivalents of the galantamine salt, preferably ≤4.5 equivalents, more preferably at 4 equivalents.

In one embodiment, Et₃N is added to the solution of galantamine hydrobromide in ACN. In one embodiment, Et₃N is added to the solution of galantamine hydrobromide in ACN at ≤5 equivalents of galantamine hydrobromide, preferably ≤4.5 equivalents, more preferably at 4 equivalents.

In one embodiment, the base is added to the solution of the galantamine salt in the organic solvent over at least 15 minutes, such as 15, 16, 17, 18, 19, 20, 21, 22, 23, 24, 25, 26, 27, 28, 29 or 30 minutes. In one embodiment, Et₃N is added to the solution of galantamine hydrobromide in ACN over at least 15 minutes.

In one embodiment, the nucleophilic catalyst is added to the mixture of the solution of the galantamine salt in the organic solvent and the base. In one embodiment, a solution of the nucleophilic catalyst in the organic solvent is added to the mixture of the solution of the galantamine salt in the organic solvent and the base.

In one embodiment, the nucleophilic catalyst is added to the mixture of the solution of the galantamine salt in the organic solvent and the base at ≤0.3 equivalents of the galantamine salt, preferably ≤0.2 equivalents, more preferably 0.1 equivalents.

In one embodiment, the nucleophilic catalyst base is added to the mixture of the solution of the galantamine salt in the organic solvent and the base at ≤0.3 equivalents of the galantamine salt, such as 0.3, 0.29, 0.28, 0.27, 0.26, 0.25, 0.24, 0.23, 0.22, 0.21, 0.2, 0.19, 0.18, 0.17, 0.16, 0.15, 0.14 0.13, 0.12, 0.11, 0.1, 0.09, 0.08, 0.07, 0.06 or 0.05 equivalents.

In one embodiment, the nucleophilic catalyst is 4-dimethylaminopyridine (DMAP) and is added to mixture of the solution of the galantamine salt in the organic solvent and the base. In one embodiment, the nucleophilic catalyst is DMAP and is added to the mixture of the solution of the galantamine salt in the organic solvent and the base at ≤0.3 equivalents of the galantamine salt, preferably ≤0.2 equivalents, more preferably 0.1 equivalents.

In one embodiment, DMAP is added to the mixture of the solution of galantamine hydrobromide in ACN and Et₃N. In one embodiment, a solution of DMAP in ACN is added to the mixture of the solution of galantamine hydrobromide in ACN and Et₃N. In one embodiment, a solution of DMAP in ACN is added to the mixture of the solution of galantamine hydrobromide in ACN and Et₃N at ≤0.3 equivalents of the galantamine salt, preferably ≤0.2 equivalents, more preferably 0.1 equivalents.

In one embodiment, a solution of DMAP in 0.3 vol ACN is added to the mixture of the solution of galantamine hydrobromide in ACN and Et₃N at ≤0.3 equivalents of the galantamine salt, preferably ≤0.2 equivalents, more preferably 0.1 equivalents.

In one embodiment, the mixture of the nucleophilic catalyst, the galantamine salt and base in the organic solvent is cooled to ≤5° C., preferably ≤3° C., more preferably 0° C.±3° C. In one embodiment, the mixture of the nucleophilic catalyst, the galantamine salt and base is cooled to s 5° C., such as 5, 4.5, 4, 3.5, 3, 2.5, 2, 1.5, 1, 0.5, 0, −0.5, −1, −1.5, −2, −2.5, −3, −3.5, −4, −4.5 or −5° C. In one embodiment, the mixture of the nucleophilic catalyst, the galantamine salt and base in the organic solvent is cooled to ≤5° C., preferably ≤3° C., more preferably 0° C.±3° C. over not less than 30 minutes, such as 30, 31, 32, 33, 34, 35, 36, 37, 38, 39, 40, 41, 42, 43, 44 or 45 min.

In one embodiment, the mixture of DMAP, galantamine hydrobromide and Et₃N in ACN is cooled to ≤5° C., preferably ≤3° C., more preferably 0° C.±3° C. In one embodiment, the mixture of DMAP, galantamine hydrobromide and Et₃N in ACN is cooled to ≤5° C., preferably ≤3° C., more preferably 0° C.±3° C. over not less than 30 minutes, such as 30, 31, 32, 33, 34, 35, 36, 37, 38, 39, 40, 41, 42, 43, 44 or 45 min.

In one embodiment, the benzoyl halide is added at ≤2 equivalents of the galantamine salt, preferably wherein the benzoyl halide is added at ≤1.5 equivalents of the galantamine salt, more preferably wherein the benzoyl halide is added at 1.2±0.5 equivalents of the galantamine salt. In one embodiment, the benzoyl halide is added at ≤2 equivalents of the galantamine salt, such as 1.15, 1.16, 1.17, 1.18, 1.19 1.2, 1.21, 1.22, 1.23, 1.24, 1.25, 1.26, 1.27, 1.28, 1.29, 1.3, 1.35, 1.4, 1.45, 1.5, 1.55, 1.6, 1.65, 1.7, 1.75, 1.8, 1.85, 1.9, 1.95 or 2 equivalents.

In one embodiment, benzoyl chloride is added at ≤2 equivalents of galantamine hydrobromide, preferably at ≤1.5 equivalents, more preferably at 1.2±0.5 equivalents.

In one embodiment, the benzoyl halide is added at an overall amount of ≤2 equivalents of the galantamine salt, preferably wherein the benzoyl halide is added at an overall amount of ≤1.5 equivalents of the galantamine salt, more preferably wherein the benzoyl halide is added at an overall amount of 1.2±0.5 equivalents of the galantamine salt.

In one embodiment, benzoyl chloride is added at an overall amount of ≤2 equivalents of galantamine hydrobromide, preferably at ≤1.5 equivalents, more preferably at 1.2±0.5 equivalents.

In one embodiment the addition of benzoyl halide to galantamine salt occurs at ≤20° C., preferably at ≤14° C., more preferably at ≤10° C., more preferably at 0±5° C.

In one embodiment
a. the organic solvent is acetonitrile (ACN),
b. the nucleophilic catalyst is 4-dimethylaminopyridine (DMAP),
c. the base is triethylamine (Et₃N) and/or
d. the benzoyl halide is added in isopropyl acetate (IPAc).

In one embodiment, ACN has a water content of ≤0.04%, such as 0.04, 0.03, 0.02, 0.01 or 0%.

In one embodiment, the method comprises the addition of benzoyl chloride to galantamine hydrobromide in the presence of a nucleophilic catalyst and a base, wherein the benzoyl chloride is added at 1.15 to ≤1.75 equivalents of the galantamine hydrobromide, preferably at 1.2±0.5 equivalents, at ≤14° C., preferably at −10 to 10° C., more preferably at 0±5° C.

In one embodiment, the method comprises the addition of benzoyl chloride to galantamine hydrobromide in the presence of a nucleophilic catalyst and a base, wherein the benzoyl chloride is added at 1.15 to ≤1.75 equivalents of the galantamine hydrobromide, such as 1.15, 1.16, 1.17, 1.18, 1.19 1.2, 1.21, 1.22, 1.23, 1.24, 1.25, 1.26, 1.27, 1.28, 1.29, 1.3, 1.35, 1.4, 1.45, 1.5, 1.55, 1.6, 1.65, 1.7 or 1.75 equivalents, at ≤14° C., such as 14, 13, 12, 11, 10, 9, 8, 7, 6, 5, 4, 3, 1.5, 2, 1.5, 1, 0.5, 0, −0.5, −1, −1.5, −2, −2.5, −3, −4 or −5° C.

In one embodiment, the addition of a benzoyl halide to a galantamine salt to form a mixture occurs at a pre-determined temperature of ≤20° C., preferably at ≤14° C., more preferably at ≤10° C., more preferably at 0±5° C., and said mixture is stirred for 60-180 minutes at said pre-determined temperature, and subsequently warmed to an ambient temperature and stirred for 3-18 hours, followed by optional washing and drying.

In one embodiment, the addition of a benzoyl halide to a galantamine salt to form a mixture occurs at a pre-determined temperature of ≤20° C., such as 20, 19, 18, 17, 16, 15, 14, 13, 12, 11, 10, 9, 8, 7, 6, 5, 4, 3, 1.5, 2, 1.5, 1, 0.5, 0, −0.5, −1, −1.5, −2, −2.5, −3, −4 or −5° C., and said mixture is stirred for 60-180 minutes at said pre-determined temperature, such as 60, 65, 70, 75, 80, 85, 90, 95, 100, 110, 120, 130, 140, 150, 160, 170 or 180 minutes, and subsequently warmed to an ambient temperature and stirred for 3-18 hours, such as 3, 4, 5, 6, 7, 8, 9, 10, 11, 12, 13, 14, 15, 16, 17 or 18 hours, followed by optional washing and drying.

In one embodiment, one half of the overall amount of the benzoyl halide is added to a galantamine salt to form a mixture at a pre-determined temperature of ≤20° C., preferably at ≤14° C., more preferably at ≤10° C., more preferably at 0±5° C., and said mixture is stirred for 60-180 minutes at said pre-determined temperature, subsequently warmed to an ambient temperature and stirred for ≥4 hours, subsequently cooled to ≤14° C., preferably −10 to 10° C., more preferably 0±3° C., and subsequently the second half of the overall amount of benzoyl halide is added to the mixture at a pre-determined temperature of ≤20° C., preferably at ≤14° C., more preferably at ≤10° C., more preferably at 0±5° C., and said mixture is stirred for 60-180 minutes at said pre-determined temperature, subsequently warmed to an ambient temperature and stirred for ≥2 hours, such as 2, 2.5, 3, 3.5, 4, 4.5, 5, 5.5, 6, 6.5, 7, 7.4 or 8 hours, followed by optional washing and drying.

In one embodiment, 0.6 equivalents of the benzoyl halide is added to a galantamine salt to form a mixture at a pre-determined temperature of ≤20° C., preferably at ≤14° C., more preferably at ≤10° C., more preferably at 0±5° C., and said mixture is stirred for 60-180 minutes at said pre-determined temperature, subsequently warmed to an ambient temperature and stirred for ≥4 hours, subsequently cooled to ≤14° C., preferably −10 to 10° C., more preferably 0±3° C., and subsequently 0.6 equivalents of benzoyl halide is added to the mixture at a pre-determined temperature of ≤20° C., preferably at ≤14° C., more preferably at ≤10° C., more preferably at 0±5° C., and said mixture is stirred for 60-180 minutes at said pre-determined temperature, subsequently warmed to an ambient temperature and stirred for ≥2 hours, followed by optional washing and drying.

In one embodiment, the addition of the benzoyl halide to form a mixture occurs over a time period of not less than 60 minutes, such as 60, 65, 70, 75, 80, 85, 90, 95, 100, 110, 120, 130, 140, 150, 160, 170 or 180 minutes. In one embodiment, the addition of one half and the second half of the overall amount of benzoyl halide to form a mixture occurs over a time period of not less than 60 minutes.

In one embodiment, after addition of the overall amount of benzoyl halide, stirring for 60-180 minutes, warming to an ambient temperature and stirring for ≥2 hours the mixture comprises ≤3% of the benzoyl halide, such as 3, 2.5, 2, 1.5, 1, 0.5 or 0%.

As outlined in the examples below, the process-related impurities MW 887 and MW 481 are typically generated in the presence of an excess amount of benzyl chloride (BzCl). However, when the amount of BzCl used within the process is too low, the reaction becomes substantially slower, resulting in an ineffective and uneconomic manufacturing process.

Thus, in the present invention, the equivalent of BzCl added within the process has been optimized with regards to controlling the amount of impurities generated at a low level and the reaction time is short.

Further, it was found that by slow addition, preferably at low temperature, the formation of process-related impurities MW 887 and MW 481 can advantageously be further reduced and controlled.

The present invention therefore provides an optimized method of benzgalantamine synthesis, in which the precise control of the amount of BzCl added to the galantamine salt (equivalents of BzCl relative to galantamine salt), preferably in combination with a controlled temperature of the reaction (target temperature of 0 deg C., within the ranges exemplified below), leads to unexpectedly beneficial avoidance or reduction of process-related impurities (for example MW 887 and MW 481). In embodiments, the control of the esterification of galantamine salt (hydrobromide) to benzgalantamine is achieved by controlling the amount of BzCl, the temperature, the time of mixing, time of temperature change, and/or the addition of BzCl over two steps, is achieved. Until the present invention, it was unknown that process-related impurities (for example MW 887 and MW 481) were generated through excess BzCl and/or higher reaction temperatures. Thus, the identification of these impurities, and optimization of the process to reduce or avoid such impurities, represents a beneficial effect, that would not have been derivable from the prior art.

In one embodiment, the addition of benzoyl halide to the galantamine salt is performed under nitrogen atmosphere. By performing the process of benzgalantamine production under nitrogen atmosphere, advantageously, the formation of N-oxide of benzgalantamine can be reduced or avoided. Performing the reaction under nitrogen atmosphere thus advantageously results in a highly pure reaction product and higher yields, as less of the reactants are consumed in unwanted side reactions.

In one embodiment, the optional washing and drying steps comprise the addition of isopropyl acetate (IPAc), followed by filtering and washing with IPAc.

In one embodiment, the optional washing and drying steps additionally comprise a subsequent distillation step, preferably a vacuum distillation step. In one embodiment the distillation step is repeated.

In one embodiment, the optional washing and drying steps additionally comprise the addition of water after distillation, preferably followed by stirring at a predetermined temperature of 20° C. 10° C. for not less than 4 h.

In one embodiment, the optional washing and drying steps additionally comprise subsequent washing by the addition of (saturated) sodium bicarbonate resulting in the separation of an organic phase, followed by extraction of the organic phase.

In one embodiment, the filtrate is subsequently concentrated by a distillation step, followed by addition of n-heptane. In one embodiment, the distillation and addition of n-heptane is repeated.

In one embodiment, the product comprises less than 2% IPAc after distillation and n-heptane addition.

In one embodiment, the distillation and addition of n-heptane is followed by filtration and washing with n-heptane. In one embodiment, a filter cake is obtained after filtration, and the filter cake is dried under vacuum.

In one embodiment, the product obtained after preparation, washing and drying has a loss on drying of ≤1%, such as 1, 0.9, 0.8, 0.7, 0.6, 0.5, 0.4, 0.3, 0.2, 0.1 or 0%.

In one embodiment, the product obtained after preparation, washing and drying is a pale brown solid.

In one embodiment, the product obtained after preparation, washing and drying comprises benzgalantamine at a purity of at least 90%, more preferably at least 95%, even more preferably at least 97%. In one embodiment, the product obtained after preparation, washing and drying comprises benzgalantamine at a purity of at least 90%, such as 90, 91, 92, 93, 94, 95, 96, 97, 98, 99 or 100%.

In one embodiment, the galantamine salt comprises an amount of epi-galantamine ≤0.3%, preferably ≤0.2%, such as 0.3, 0.25. 0.2, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01 or 0%. In one embodiment, the galantamine hydrobromide comprises an amount of epi-galantamine ≤0.3%, preferably ≤0.2%.

In one embodiment, the galantamine salt comprises an amount of (−)-galantamine ≤0.15%, preferably ≤0.1% such as 0.15, 0.1, 0.09, 0.08, 0.07, 0.06, 0.05, 0.04, 0.03, 0.02, 0.01 or 0%. In one embodiment, galantamine salt hydrobromide an amount of (−)-galantamine ≤0.15%, preferably ≤0.1%.

In one embodiment, the product obtained after preparation, washing and drying comprises (+)-benzgalantamine at an enantiomeric purity of at least 90%, such as 90, 91, 92, 93, 94, 95, 96, 97, 98, 99 or 100%.

The method of the present invention also provides the advantage of control over stereoisomeric and/or enantiomeric purity. By limiting the amount of (−)-galantamine and epi-galantamine within the galantamine salt, the content of (−)-Benzgalantamine gluconate and impurities related to epi-Galantamine such as epi-Galantamine benzoylester can be controlled and limited. As demonstrated in examples below, the content of epi-galantamine related impurities can be limited to 0.15%. Consequently, the present invention allows for the production of benzgalantamine in an enantiomerically and stereoisomerically pure form, specifically as (+)-Benzgalantamine.

In one embodiment, the galantamine salt comprises an amount of lycoramine ≤0.1% when assessed by the area under the curve from an HPLC analysis of the galantamine salt, preferably ≤0.08%, such as 0.08, 0.07, 0.06, 0.05, 0.04, 0.03, 0.02, 0.01 or 0%. In one embodiment, galantamine hydrobromide comprises an amount of lycoramine ≤0.1% when assessed by the area under the curve from an HPLC analysis of galantamine hydrobromide, preferably ≤0.08%.

In one embodiment, the galantamine salt comprises an amount of O-desmethyl-galantamine of ≤0.2% when assessed by the area under the curve from an HPLC analysis of the galantamine salt, preferably ≤0.15%, such as 00.2, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01 or 0%. In one embodiment, galantamine hydrobromide comprises an amount of O-desmethyl-galantamine ≤0.2% when assessed by the area under the curve from an HPLC analysis of galantamine hydrobromide, preferably ≤0.15%.

In one embodiment, the galantamine salt comprises an amount of N-oxide of galantamine of ≤0.3% when assessed by the area under the curve from an HPLC analysis of the galantamine salt, preferably ≤0.2%, such as 0.3, 0.25, 0.2, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01 or 0%. In one embodiment, galantamine hydrobromide comprises an amount of N-oxide of galantamine ≤0.3% when assessed by the area under the curve from an HPLC analysis of galantamine hydrobromide, preferably ≤0.2%.

In one embodiment, the galantamine salt comprises an amount of N-desmethyl-galantamine of ≤0.3% when assessed by the area under the curve from an HPLC analysis of the galantamine salt, preferably ≤0.2%, such as 0.3, 0.25, 0.2, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01 or 0%. In one embodiment, galantamine hydrobromide comprises an amount of N-desmethylgalantamine ≤0.3% when assessed by the area under the curve from an HPLC analysis of galantamine hydrobromide, preferably ≤0.2%.

By limiting the amount of epi-galantamine, lycoramine, 0-desmethyl-galantamine, N-oxide of galantamine and N-desmethylgalantamine within the galantamine salt, preferably galantamine hydrobromide, used within the process of the present invention, the amount of corresponding impurities, which at least partially cannot be removed by subsequent purification, can advantageously be effectively controlled and limited at an acceptable amount during the process, resulting in a highly pure product. This advantageously increased the yield of the method described herein, as less reactant consuming side reactions occur.

In one embodiment, the process additionally comprises the formation of a salt of benzgalantamine, preferably subsequent to the process according to the preceding embodiments.

In one embodiment, the process comprises additionally the addition of a gluconolactone, preferably in water, to benzgalantamine as prepared according to any one of the preceding embodiment, preferably in methyl ethyl ketone (MEK), to prepare a gluconate salt of benzgalantamine.

In one embodiment, the process comprises additionally the addition of a gluconolactone in water, to benzgalantamine as prepared according to any one of the preceding embodiment in methyl ethyl ketone (MEK), prepare a gluconate salt of benzgalantamine, preferably at an MEK/H₂O ratio (vol./vol) between 10/1 to 15/1, such as 10/1, 10.5/1, 11/1, 11.5/1, 12/1, 12.5/1, 13/1, 13.5/1, 14/1, 14.5/1 or 15/1.

As shown in the examples below, within this range of MEK/H₂O ratios the crystalline form of the reaction product can advantageously be well controlled, preferably the reaction product is provided as Form A at these MEK ratios. By controlling the crystalline form of the reaction product within the process, advantageously, no additional recrystallization step after product formation is necessary.

In one embodiment, the mixture of a gluconolactone and benzgalantamine in water and MEK is stirred to form an emulsion. In one embodiment, a seed of the benzgalantamine salt is added to the emulsion, preferably a 0.4% seed of the benzgalantamine salt in MEK. In one embodiment, the addition of the seed and subsequent stirring is repeated until precipitation occurs.

In one embodiment, after precipitation the mixture is stirred at a predetermined temperature of 10 to 30° C., preferably 15 to 25° C., for ≥12 hours, such as 12, 13, 14, 15, 16, 17, 18, 19 or 20 hours.

In one embodiment, the volume of the mixture is subsequently reduced by distillation. In one embodiment, the process of the present invention comprises a distillation subsequent to the formation of a salt of benzgalantamine, wherein the distillation is preferably an azeotropic distillation. By employing azeotropic distillation instead of addition of MEK as anti-solvent for concentration of the mixture, advantageously more product could be obtained. By using azeotropic distillation the yield was improved while other product specifications of the product such as the polymorphic form were not changed. Further, no degradation of the product was observed during azeotropic distillation, thus advantageously not impairing stability of the product.

In one embodiment, the water content of the mixture after the second distillation step is ≤4%, such as 4, 3.5, 3, 2.5, 2, 1.5, 1, 0.5 or 0%.

In one embodiment, the second distillation step is followed by stirring, followed by filtering to obtain a filter cake and washing the obtained filter cake with MEK.

In one embodiment, the filter cake is dried, preferably vacuum dried. In one embodiment, the dried filter cake is free of MEK. In one embodiment the dried filter cake has a water content of ≤9%, such as 9, 8, 7, 6, 5, 4, 3, 2, 1 or 0%.

In one embodiment, the filter cake comprises ≤4000 ppm of MEK after the additional drying step.

In one embodiment, the salt of benzgalantamine is benzgalantamine gluconate.

In one embodiment, benzgalantamine gluconate is in crystalline form A (anhydrate).

In one embodiment, benzgalantamine gluconate is in crystalline form A (anhydrate), wherein said crystalline form A has prominent peaks at 3.61, 10.98, 14.41 and 18.44 degrees 2-theta (±0.2) in a powder X-ray diffraction pattern.

These 4 peaks are selected from the prominent peak list provided below and appear to exhibit no substantial overlap with prominent peaks in the XRPD patterns for Forms B-D, or Materials E-G, as disclosed in WO 2022/150917 (incorporated by reference). In one embodiment, Form A can therefore be reliably distinguished using one or more prominent peaks, for example as mentioned above, upon comparison of the corresponding powder X-ray diffraction patterns.

In one embodiment, Form A has one or more additional prominent peaks at 15.20, 17.31, 17.79, 22.77, 23.64, 24.88 and 34.31 degrees 2-theta (±0.2) in a powder X-ray diffraction pattern.

These peaks are selected from the prominent peak list and appear to exhibit no substantial overlap with prominent peaks in the XRPD patterns for Forms B-D, or Materials E-G.

In one embodiment, Form A has at least five prominent peaks selected from the list consisting of 3.61, 10.98, 13.80, 14.41, 14.56, 15.08, 15.20, 17.02, 17.31, 17.79, 18.44, 19.24, 20.18, 20.91, 21.22 and 22.40 degrees 2-theta (±0.2) in a powder X-ray diffraction pattern.

Typically, not all peaks from this list need be detected in order to determine the presence of Form A in any given synthesis product, preparation or composition. According to the invention, for example in some embodiments, 1, 2, 3, 4, 5, 6, 7, 8, 9, 10 or more peaks, preferably those with relatively high signal intensity, may be employed to determine any given crystal form. For example, the 4, 5, 6, 7, 8, 9 or 10 most intense peaks may be employed to identify any given crystal form. In one embodiment, sufficient identification of any given crystal form, such as Form A, is achieved when the presence of at least three or four prominent peaks can be determined based on XRPD comparisons.

Typically, prominent XRPD peaks are the strongest low angle, non-overlapping peaks observed in a XRPD pattern. In some embodiments, the “prominent peaks” have preferably a ≥20% relative intensity, preferably ≥30% relative intensity, more preferably ≥40% relative intensity, in a powder X-ray diffraction pattern. The values of relative intensity may however vary depending on device or analysis mode and are not inherently limiting to the solid forms described herein.

In one embodiment, Form A has peaks at 7.25 and/or 12.67 degrees 2-theta (±0.2) in a powder X-ray diffraction pattern. These peaks are of relatively low intensity compared to the peaks outlined above as predominant peaks. However, peaks at 7.25 and/or 12.67 degrees 2-theta appear to be absent in all other patterns for Forms B-D or Materials E-G.

In one embodiment, the peaks are determined using powder X-ray diffraction analysis in transmission mode.

In one embodiment, Form A has at least three peaks selected from the list consisting of 10.98, 14.41, 17.31, 18.44 and 22.40 degrees 2-theta (±0.2) in a powder X-ray diffraction pattern. In one embodiment, said three peaks are within the five peaks with the highest relative intensity in a powder X-ray diffraction pattern obtained using analysis in transmission mode. In one embodiment, these five peaks are the most intense peaks in the XRPD pattern using transmission mode, as outlined in the examples below.

In one embodiment, the peaks are determined using powder X-ray diffraction analysis in reflectance mode.

In one embodiment, Form A has at least three peaks selected from the list consisting of 3.61, 7.25, 10.98, 14.56 and 22.40 degrees 2-theta (±0.2) in a powder X-ray diffraction pattern. In one embodiment, said three peaks are preferably within the five peaks with the highest relative intensity in a powder X-ray diffraction pattern obtained using analysis in reflectance mode. In one embodiment, these five peaks are the most intense peaks in the XRPD pattern using reflectance mode, as outlined in the example below.

In one embodiment, Form A has one or more peaks selected from the list consisting of 3.61, 7.25, 10.98, 14.56, 22.40 degrees 2-theta (±0.2) in a powder X-ray diffraction pattern. These peaks are also observable from the XRPD pattern using reflectance mode.

In one embodiment, Form A has one or more doublets selected from the list consisting of 14.41 and 14.56, 15.08 and 15.20, and 24.88 and 25.09 degrees 2-theta (±0.2) in a powder X-ray diffraction pattern. These doublets may be used to identify Form A and optionally distinguish the Form from other forms.

Provided below is a Table of the typically observed XRPD pattern peaks for Form A collected in transmission mode.

Peak list Form A: Peak list determined from the powder X-ray diffraction pattern of Form A, according to FIG. 9. Accuracy of degrees 2-theta is provided at 2 decimal points, some variation dependent on batch or device may be evident.

| 2θ (°) | d (Å) | I (%) |
|---|---|---|
| 3.61* | 24.5 | 47 |
| 7.25 | 12.2 | 18 |
| 10.52 | 8.40 | 14 |
| 10.98* | 8.05 | 100 |
| 11.71 | 7.55 | 4 |
| 12.67 | 6.98 | 23 |
| 13.46 | 6.57 | 10 |
| 13.80* | 6.41 | 45 |
| 14.41* | 6.14 | 71 |
| 14.56* | 6.08 | 52 |
| 15.08* | 5.87 | 40 |
| 15.20* | 5.82 | 46 |
| 16.16 | 5.48 | 25 |
| 16.44 | 5.39 | 22 |
| 17.02* | 5.20 | 46 |
| 17.31* | 5.12 | 66 |
| 17.79* | 4.98 | 41 |
| 18.24 | 4.86 | 8 |
| 18.44* | 4.81 | 67 |
| 19.24* | 4.61 | 56 |
| 19.43 | 4.56 | 10 |
| 19.80 | 4.48 | 24 |
| 20.18* | 4.40 | 40 |
| 20.91* | 4.24 | 64 |
| 21.22* | 4.18 | 57 |
| 21.54 | 4.12 | 19 |
| 22.09 | 4.02 | 19 |
| 22.40* | 3.97 | 86 |
| 22.77* | 3.90 | 41 |
| 23.64* | 3.76 | 39 |
| 24.30 | 3.66 | 13 |
| 24.88* | 3.58 | 41 |
| 25.09* | 3.55 | 44 |
| 25.44 | 3.50 | 9 |
| 25.76 | 3.46 | 15 |
| 25.89 | 3.44 | 25 |
| 26.37 | 3.38 | 12 |
| 26.62 | 3.35 | 5 |
| 26.91 | 3.31 | 5 |
| 27.19 | 3.28 | 17 |
| 27.37 | 3.26 | 19 |
| 27.82 | 3.20 | 23 |
| 27.99 | 3.18 | 35 |
| 28.95 | 3.08 | 14 |
| 29.34 | 3.04 | 11 |
| 29.83 | 2.99 | 21 |
| 30.37 | 2.94 | 15 |
| 30.92 | 2.89 | 17 |
| 31.68 | 2.82 | 6 |
| 32.44 | 2.76 | 9 |
| 33.39 | 2.68 | 5 |
| 34.31 | 2.61 | 34 |

*Peaks may in some embodiments be considered as prominent peaks observed in the XRPD pattern.

In one embodiment, Form A exhibits an onset of melting at a temperature of 107-121° C., preferably 107.8 to 120.2° C., when assessed using differential scanning calorimetry (DSC).

In one embodiment, Form A exhibits a weight loss of <1%, preferably <0.5%, more preferably less than <0.3%, or <0.2%, prior to the onset of melt using DSC when assessed using Thermo-Gravimetric Analysis (TGA).

In one embodiment, Form A exhibits a solubility in water of above 100 mg/mL, preferably above 120 mg/mL, more preferably about 123 mg/mL. Methods for determining solubility in water are known to a skilled person and may be carried out without undue burden.

In one embodiment, Form A is stable and shows no or negligible conversion to any one of Forms B-D or Materials E-G when stored at a relative humidity (RH) of less than 75%, preferably less than 50%, more preferably Form A is stable at an RH of about 43% or less.

Form A represents an advantageous form of the Benzgalantamine gluconate salt. Due to its high stability when stored under low humidity and good solubility in water, it represents an ideal form for preparation of a pharmaceutical agent and to be used in formulation and manufacture of drug products.

In one embodiment, benzgalantamine gluconate is in crystalline form B (tetrahydrate).

In one embodiment, benzgalantamine gluconate is in crystalline form B, wherein said crystalline form has prominent peaks at 10.69, 17.17, 21.00 and 24.67 degrees 2-theta (±0.2) in a powder X-ray diffraction pattern.

These 4 peaks are selected from the prominent peak list provided in WO 2022/150917 (incorporated by reference) and appear to exhibit no substantial overlap with prominent peaks in the XRPD patterns for Forms A, C or D or Materials E-G, as disclosed in WO 2022/150917. In one embodiment, Form B can therefore be reliably distinguished using one or more prominent peaks, as disclosed in WO 2022/150917.

In one embodiment, benzgalantamine gluconate is in crystalline form C (hemihydrate/monohydrate).

In one embodiment, benzgalantamine gluconate is in crystalline form C, wherein said crystalline form has prominent peaks at 3.90, 9.74, 10.35 and 21.43 (also optionally 15.66 and/or 23.90) degrees 2-theta (±0.2) in a powder X-ray diffraction pattern.

These peaks are selected from the prominent peak list provided in WO 2022/150917 (incorporated by reference) and appear to exhibit no substantial overlap with prominent peaks in the XRPD patterns for Forms A, B or D or Materials E-G, as disclosed in WO 2022/150917. In one embodiment, Form C can therefore be reliably distinguished using one or more prominent peaks, as disclosed in WO 2022/150917.

In one embodiment, benzgalantamine gluconate is in crystalline form D (dihydrate).

In one embodiment, benzgalantamine gluconate is in crystalline form D, wherein said crystalline form has prominent peaks at 3.76, 10.16, 14.77 and 19.03 (also optionally 17.96, 18.86 and/or 28.14) degrees 2-theta (±0.2) in a powder X-ray diffraction pattern.

These peaks are selected from the prominent peak list provided in WO 2022/150917 (incorporated by reference) and appear to exhibit no substantial overlap with prominent peaks in the XRPD patterns for Forms A, B or C or Materials E-G, as disclosed in WO 2022/150917. In one embodiment, Form D can therefore be reliably distinguished using one or more prominent peaks, as disclosed in WO 2022/150917.

In one embodiment, benzgalantamine gluconate is in an amorphous form.

Amorphous solids normally unveil a featureless XRD pattern with broad, diffuse halos because of the absence of the long-range order of repeating crystal lattice.

In embodiments, heating material of Form A to just beyond the melt (about 125° C.) and subsequent cooling can result in an amorphous material. In embodiments, the amorphous form can be generated by slow evaporation of benzgalantamine gluconate from methanol. In embodiments, the glass transition temperature of the amorphous solid was determined to be near 41° C.

In embodiment of the invention, the synthesis of benzgalantamine as described herein may be carried out, and optionally followed by production of a gluconate salt, and optionally followed by subsequent generation of any one of solid forms A, B, C or the amorphous form, for example, as described in WO 2022/150917.

In one aspect, the invention relates to a composition comprising or consisting of a reaction product prepared by the process according to the present invention.

In one embodiment, the reaction product comprises one or more of the following impurities, preferably at low amounts or negligible amounts;

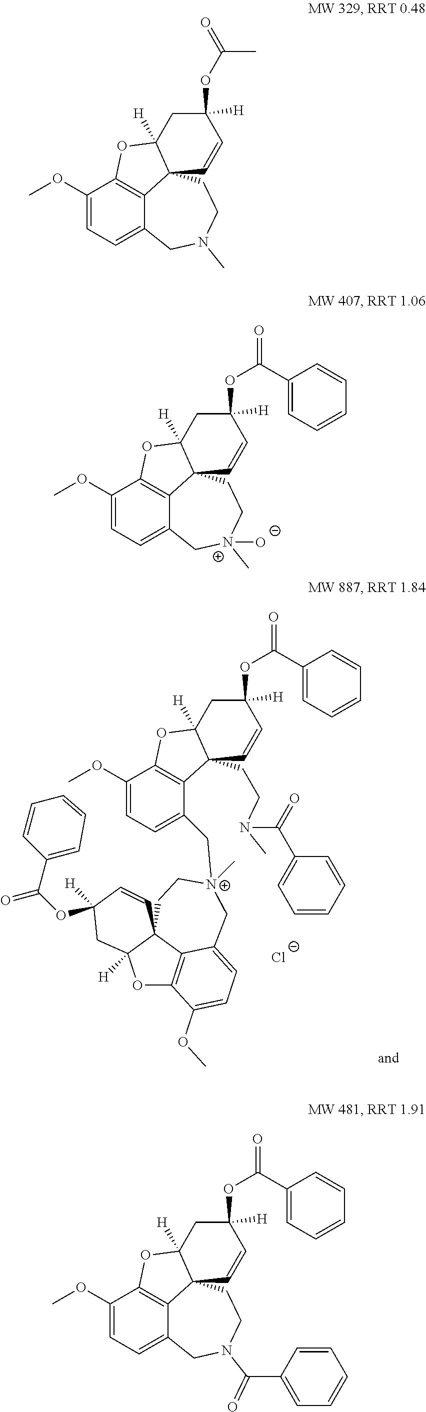

In one embodiment, the reaction product does not comprise one or more impurities selected from MW329, MW407, MW887 and/or MW481. In such embodiments, the one or more of MW329, MW407, MW887 and/or MW481 are either not present or not detectable using detection means described herein.

In one embodiment, the reaction product prior to salt formation comprises one or more of the impurities according to the preceding embodiment, preferably at negligible amounts.

In one embodiment, the reaction product after salt formation comprises one or more of the impurities according to the preceding embodiment, preferably at negligible amounts.

In one embodiment, the reaction product comprises one or more of the one or more impurities present at an amount of ≤3.5% when assessed by the area under the curve from an HPLC analysis of the reaction product, preferably at ≤3%, more preferably at ≤2.6%, such as 3.5, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1 or 0%.

In one embodiment, the reaction product comprises one or more of the following impurities present at an amount of ≤1% when assessed by the area under the curve from an HPLC analysis of the reaction product, preferably ≤0.5%, more preferably ≤0.1%, even more preferably ≤0.08%. In one embodiment, the reaction product comprises one or more of the following impurities present at a total amount of ≤1% when assessed by the area under the curve from an HPLC analysis of the reaction product, preferably ≤0.5%, more preferably ≤0.1%, even more preferably ≤0.08%.

In one embodiment, the reaction product comprises at least one of the following two impurities:

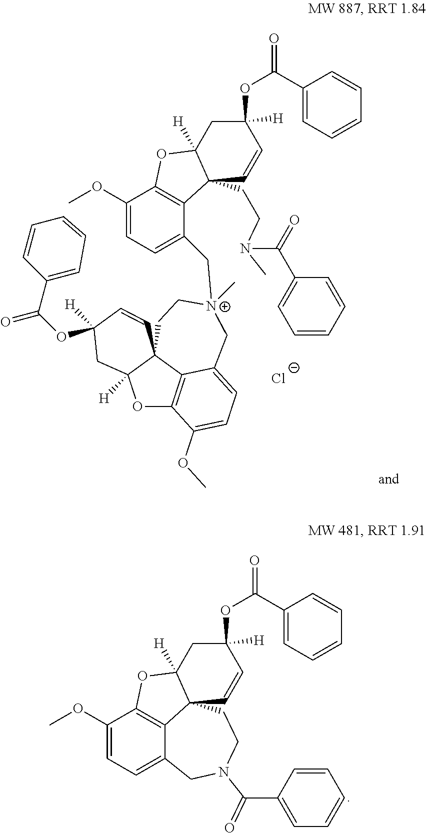

In embodiments, these impurities may be present at an amount of ≤3.5%, preferably ≤3%, more preferably ≤2%, of the material produced by the process of the present invention when assessed by the area under the curve from an HPLC analysis of the reaction product, such as 3.5, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1 or 0%.

In one embodiment, the reaction product comprises one or more of the two impurities present at an amount of ≤1% when assessed by the area under the curve from an HPLC analysis of the reaction product, preferably ≤0.5%, more preferably ≤0.1%, even more preferably ≤0.08%. In one embodiment, the reaction product comprises one or more of the following impurities present at a total amount of ≤1% when assessed by the area under the curve from an HPLC analysis of the reaction product, preferably ≤0.5%, more preferably ≤0.1%, even more preferably ≤0.08%.

In one embodiment, the one or more impurities are removed from the reaction product by purification. In one embodiment, the reaction product is purified after preparation by the process of the present invention. In one embodiment, the purified reaction product is free from the one or more impurities or comprises only a negligible amount of the impurities.

Advantageously in the process of the present invention, process-related impurities, preferably the process-related impurities outlined above, can be controlled at negligible amounts within the material produced by the process. This advantageously allows the highly controlled production of a highly pure product with high yields.

The one or more impurities described herein may be detected in a product of synthesis, i.e. in a preparation of benzgalantamine, or in a pharmaceutical composition comprising benzgalantamine. Detecting one or more impurities, for example at the amounts described herein, may indicate that the method of the invention has been employed.

In one embodiment, the reaction product has a particle size D(v, 0.1) of 10 to 20 μm, preferably 11 to 17 μm, more preferably 11 to 16 μm, such as 10, 11, 12, 13, 14, 15, 16, 17, 18, 19 or 10 μm.

In one embodiment, the reaction product has a particle size D(v, 0.5) of 30 to 50 μm, preferably 35 to 46 μm, more preferably 37 to 46 μm, such as 30, 31, 32, 33, 34, 35, 36, 37, 38, 39, 40, 41, 42, 43, 44, 45, 46, 47, 48, 49 or 50 μm.

In one embodiment, the reaction product has a particle size D(v, 0.9) of 70 to 90 μm, preferably 71 to 89 μm, more preferably 71 to 89 μm, such as 70, 71, 72, 73, 74, 75,76, 77, 78, 79, 80, 81, 82, 83, 84, 85, 86, 88, 89 or 90 μm.

D(v, 0.1), D(v, 0.5), and D(v, 0.9) refer to the particle size distribution within a sample based on volume. D(v, 0.1) refers to the particle diameter at which 10% of the total particles in the sample have a smaller size by volume. D(v, 0.5), also known as the median volume diameter, represents the particle diameter at which 50% of the particles in the sample have a larger size by volume and 50% have a smaller size by volume. D(v, 0.9) refers to the particle diameter at which 90% of the particles in the sample have a smaller size by volume. These values are commonly determined using laser diffraction or other particle size analysis techniques that measure the volume-based distribution of particle sizes within a sample. Such techniques are well known to a person skilled in the art.

Advantageously, by the process of the present invention, the reaction product can be provided in a narrow size distribution and particle size that is suitable for further processing such as the manufacture of a pharmaceutical composition without the need for further means such as milling.

In one aspect, the invention relates to a pharmaceutical composition comprising a composition or reaction product according to the present invention together with a pharmaceutically acceptable carrier.

Pharmaceutical acceptable carriers are known to a skilled person and may be selected depending on, for example, medical indication, patient, administration route, dose and formulation.

In one embodiment, the pharmaceutical composition of the invention and/or the preparation of the invention as described herein, is packaged to reduce atmospheric moisture in contact with said composition. Suitable packaging is known to a skilled person. In some embodiments, aluminium foil blister packaging (Alu-Alu), packaging with polymeric films with aluminium layers(s) and/or using a desiccant are employed.

In one embodiment, the pharmaceutical composition is suitable for oral administration. In embodiments, the composition is in the form of a tablet, said tablet comprising: a tablet core, wherein said core comprises the benzgalantamine or salt thereof, as produced according to the method described herein, and an enteric coating, wherein said enteric coating is configured for dissolution at pH 5.5 and above.

In embodiments, due to the enteric coating of such compositions, the release of benzgalantamine occurs in the intestine above pH 5.5, resulting in a slower and lower rise to Cmax of Galantamine derived from benzgalantamine compared to an immediate release galantamine composition, releasing galantamine in the stomach, and a higher Cmax compared to an extended-release galantamine composition.

In one embodiment, the pharmaceutical composition is suitable for oral or transmucosal administration. Preferred but non-limiting modes of transmucosal administration are selected preferably from oral cavity (e.g., sublingual and/or buccal) or intranasal administration.

In one embodiment, the pharmaceutical composition is for use in the treatment of a brain disease associated with cognitive impairment. The invention further relates to methods for treating brain disease associated with cognitive impairment comprising administering a composition or a preparation of the invention to a subject in need thereof.

In one embodiment, the brain disease is associated with a cholinergic deficit. In one embodiment, the composition is used as or for use as a nicotinic acetylcholine receptor sensitizing agent.

In one embodiment, the brain disease is selected from the group consisting of a brain disease with a cholinergic deficit, Alzheimer's disease, Parkinson's disease, dementia, schizophrenia, epilepsy, stroke, poliomyelitis, neuritis, myopathy, oxygen and nutrient deficiencies in the brain after hypoxia, anoxia, asphyxia, cardiac arrest, chronic fatigue syndrome, poisoning, anaesthesia, spinal cord disorders, central inflammatory disorders, autism, Rett's syndrome, postoperative delirium, neuropathic pain, abuse of alcohol and drugs, addictive alcohol and/or nicotine craving, traumatic brain injury, persistent post-concussion symptoms, and effects of radiotherapy.

The invention therefore also relates to a method for treating a brain disease associated with cognitive impairment in a subject, the method comprising administering a therapeutically effective amount of benzgalantamine gluconate to a subject in need thereof.

All features described in the present specification may be employed to define any other embodiment or aspect of the invention. For example, the features above with respect to the process for preparing benzgalantamine and salts thereof have a structural and functional outcome on the reaction product of the present invention, such that the reaction product, the composition and pharmaceutical composition can, in embodiments, be described by features of or derived from the process of preparing, and vice versa.

### DETAILED DESCRIPTION

### General Terms:

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

Thus, the terms “comprising”/“including”/“having” mean that any further component (or likewise features, integers, steps and the like) can/may be present. The term “consisting of” means that no further component (or likewise features, integers, steps and the like) is present.

The term “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

Thus, the term “consisting essentially of” means those specific further components (or likewise features, integers, steps and the like) can be present, namely those not materially affecting the essential characteristics of the composition, device or method. In other words, the term “consisting essentially of” (which can be interchangeably used herein with the term “comprising substantially”), allows the presence of other components in the composition, device or method in addition to the mandatory components (or likewise features, integers, steps and the like), provided that the essential characteristics of the device or method are not materially affected by the presence of other components.

The term “method” or “process” refers to manners, means, techniques and procedures for accomplishing a given task including, but not limited to, those manners, means, techniques and procedures either known to, or readily developed from known manners, means, techniques and procedures by practitioners of the chemical, biological and biophysical arts. As used herein, the “patient” or “subject” may be a vertebrate. In the context of the present invention, the term “subject” includes both humans and animals, particularly mammals, and other organisms.

### Compounds:

Galantamine ((4aS,6R,8aS)-3-Methoxy-11-methyl-4a,5,9,10,11,12-hexahydro-6H-benzofuro[3a,3,2-ef][2]benzazepin-6-ol (CAS 357-70-0)), is a reversible acetylcholinesterase inhibitor, (AChEI) and thus belongs to the group of reversible AChE inhibitors. It further enhances cholinergic activity by non-competitive, allosteric modulation of the nicotinic acetylcholine receptors (nAChR).

It is approved as immediate release tablet and oral solution and extended-release capsule and tablet and marketed as RAZADYNE® and RAZADYNE® ER (U.S.) and as REMINYL® (elsewhere).

The chemical structure of galantamine is:

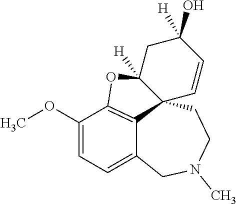

According to the full prescribing information of RAZADYNE® and RAZADYNE® ER (U.S.) and the, the regulatory approved indication is mild to moderate dementia of Alzheimer's type. According to the first approval of REMINYL in Sweden which was followed by several approvals in other European states the indication is similar to the U.S. approval for mild to moderate dementia of Alzheimer's type. RAZADYNE® tablets are available as 4 mg, 8 mg and 12 mg galantamine tablets. RAZADYNE® ER capsules are available as −8 mg, 16 mg and 24 mg extended-release capsules. The recommended initial dosage regime for RAZADYNE® 4 mg tablets is twice daily. This dose may be increased up to 12 mg twice daily. The recommended initial dose and dosage regime for RAZADYNE® ER capsules is one 8 mg capsule daily, which may be increased to 24 mg once daily.

Galantamine is well absorbed with an absolute oral bioavailability of −90% and is rapidly and completely absorbed with a tₘₐₓ of ~1 hour. Cₘₐₓ was decreased by 25% and Tₘₐₓ was delayed by 1.5 hours, when galantamine was administered with food. The mean volume of distribution of galantamine is 175 L and plasma protein binding is 18% at therapeutic conditions. In whole blood, galantamine is mainly distributed into the cellular fraction (52.7%) (RAZADYNE®, 2020). Maximum inhibition of acetylcholinesterase activity of about 40% was achieved about 1 hour after a single oral dose of 8 mg galantamine in healthy male subjects (IB third edition, May 2020). Galantamine is metabolized by hepatic cytochrome P450 (CYP) enzymes, glucuronidated, and excreted unchanged in the urine. In vitro studies indicate that CYP2D6 and CYP3A4 were the major isoenzymes involved in the metabolism of galantamine, and inhibitors of both pathways increase oral BA of galantamine modestly.

Benzgalantamine, also termed galantamine benzoate, or ALPHA-1062 ((4aS,6R,8aS)-4a,5, 9,10,11,12-hexahydro-3-methoxy-11-methyl-6H[1]benzofuro[3a,3,2-ef][2]benzazepin-6benzoate) (CAS. No.: 224169-27-1, also termed memogain or GLN-1062) is a galantamine prodrug, exhibiting no pharmacological effect on AChE activity or modulatory activity on nAChR. It is cleaved by carboxylesterases and in acidic environment, releasing one molecule of galantamine from each molecule of prodrug

The chemical structure of benzgalantamine is:

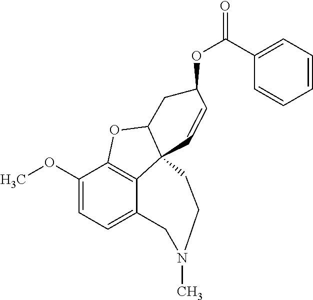

Acetylcholine (ACh) is a transmitter of the nervous system acting on postsynaptic muscarinic acetylcholine (mAChR) or nicotinic acetylcholine receptors (nAChR). ACh is cleaved by acetylcholinesterase (aChE) present in the synaptic cleft, thus inhibition of aChE and thus reduced cleavage of ACh, results in enhanced concentrations of ACh in the synaptic cleft. ACh is present in the central nervous system (CNS) and the peripheral nervous system (PNS).

Effects of ACh include mediation of contraction of skeletal muscle, regulation of the autonomic nervous system influencing for example blood pressure, heart rate, digestion and metabolism, and further regulation of reward and cognitive functions in the CNS. A deficiency of ACh (cholinergic deficit) can result in impairment of functions and effects of ACh in the somatic nervous system and autonomic nervous system and further in the CNS resulting in impairments of cognitive function (cognitive impairment), learning and memory.

Diseases and/or symptoms associated with cognitive impairment and/or with a cholinergic deficit include but are not limited to brain diseases with a cholinergic deficit, a brain disease with a cholinergic deficit, Alzheimer's disease, Parkinson's disease, dementia, schizophrenia, epilepsy, stroke, poliomyelitis, neuritis, myopathy, oxygen and nutrient deficiencies in the brain after hypoxia, anoxia, asphyxia, cardiac arrest, chronic fatigue syndrome, poisoning, anesthesia, spinal cord disorders, central inflammatory disorders, Lewy Body Disease, multiple sclerosis, skeletal muscle pain, autism, Rett's syndrome, motor neuron disease such as amyotrophic lateral sclerosis, traumatic brain injury, post-traumatic stress disorder, postoperative delirium, neuropathic pain, abuse of alcohol and drugs, addictive alcohol and/or nicotine craving, severe gas in the gastro-intestinal tract (GIT), constipation, low blood pressure, erratic heart rate, and effects of radiotherapy. These medical indications represent preferred embodiments of the medical use of the invention in the treatment of a disease and/or symptoms associated with cognitive impairment and/or with a cholinergic deficit.

Alzheimer's disease (AD), also referred to simply as Alzheimer's, is a chronic neurodegenerative disease that gradually worsens over time. It is the cause of 60-70% of cases of dementia. The most common early symptom is difficulty in remembering recent events. As the disease advances, symptoms can include problems with language, disorientation (including easily getting lost), mood swings, loss of motivation, not managing self-care, and behavioral issues.

Parkinson's disease (PD), or simply Parkinson's, is a long-term progressive degenerative disorder of the central nervous system that mainly affects the motor system. As the disease worsens, non-motor symptoms become more common. Early in the disease, the most obvious symptoms are shaking, rigidity, slowness of movement, and difficulty with walking. Thinking and behavioral problems may also occur. Dementia becomes common in the advanced stages of the disease. The main motor symptoms are collectively called “parkinsonism”, or a “parkinsonian syndrome”.

A prodrug is defined as inactive or less active agent that is transformed (also termed “metabolized”) in an organism to an active agent also termed “metabolite”. In this context, a prodrug is an inactive or less active precursor of an active drug that undergoes transformation into the active form of the drug in vivo by enzymatic cleavage or one or more non-enzymatic chemical processes in a predictable fashion. Prodrugs are developed to influence physicochemical, organoleptic, pharmacokinetic and pharmacodynamic properties of active drugs. Usually, prodrugs are characterized in that the drug is chemically coupled to a carrier molecule, which is cleaved off in the organism (carrier-bound prodrugs) resulting in transformation to the active drug. Common carrier-bound prodrugs comprise coupling of the carrier by a covalent ester linkage (ester prodrugs). Further types of prodrugs include bio precursors, which are not coupled to a carrier molecule but metabolized or conjugated directly in the organism resulting in the active drug, and co-prodrugs (mutual prodrugs), comprising two or more active drugs coupled to each other. The prodrug of the present invention benzgalantamine comprises a covalent ester linkage between the active drug galantamine and benzoate, which upon cleavage releases the active drug galantamine.

The present invention relates further to salts of benzgalantamine. In some embodiments, benzgalantamine is present as a salt of benzgalantamine, or is present in a crystalline form of benzgalantamine, or is present as a polymorph or hydrate of benzgalantamine. The terms “salt” refers to salts prepared by conventional means that include basic salts of inorganic and organic acids, including but not limited to acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, gluconate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, saccharate, succinate, tartrate, thiocyanate, tosylate and undecanoate. For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

In a preferred embodiment the salt comprises of stoichiometric and/or non-stoichiometric salts and/or hydrates of benzgalantamine, whereby the salt is preferably described as:

Benzgalantamine·n HX·m H₂O,

whereby n, m=0-5 and n and m can be the same or different, and HX=an acid, selected preferably from acetic acid, citric acid, lactic acid, gluconic acid, maleic acid or saccharic acid.

Preferred are quaternary nitrogen salts (also termed “quaternary ammonium salts” herein) of benzgalantamine with gluconic acid (gluconate salt), acetic acid (acetate salt), maleic acid (maleate salt), lactic acid (lactate salt), citric acid (citrate salt), and saccharic acid (saccharate salt).

Salts of benzgalantamine disclosed in the prior art include maleate, lactate saccharate and gluconate. In one preferred embodiment of the invention the benzgalantamine or a salt thereof is benzgalantamine gluconate.

The chemical structure of benzgalantamine (ALPHA-1062) gluconate is:

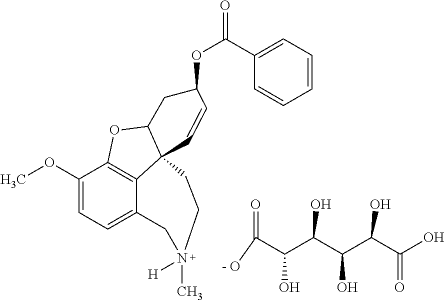

As used herein, polymorphism is the existence of a drug substance in multiple crystalline forms which exhibit differing arrangements/conformations of the molecules in a crystal lattice which can affect the solid-state properties of the material. For example, Form A as described herein may be considered a polymorph of the ALPHA-1062 gluconate.

As used herein, pseudo-polymorphism is the existence of a drug substance in a crystalline form which contains solvates/hydrates bound within a crystal lattice with the drug substance. For example, the hydrate Forms B-D may be considered pseudopolymorphs of ALPHA-1062 gluconate.

As used herein, hydrates are crystalline forms that include water molecules in their crystal lattice. In addition to polymorphs and the amorphous solid state, other examples of possible solid states are solvates and hydrates. Hydrates are frequently encountered solvates in pharmacy, because water is applied for many processing steps. APIs when exposed to water may form hydrates and hydrates may lose their water under high temperature or low humidity.

As used herein, crystalline preferably means a material that has an ordered, long range molecular structure. The degree of crystallinity of a crystal form can be determined by many techniques including, for example, powder X-ray diffraction, moisture sorption, differential scanning calorimetry, solution calorimetry, and dissolution properties.

As used herein, amorphous solids are not crystalline due to the disordered arrangements of the drug substance molecules in the solid state.

Crystalline organic compounds consist of a large number of atoms that are arranged in a periodic array in three-dimensional space. The structural periodicity normally manifests distinct physical properties, such as sharp, explicit spectral features by most spectroscopic probes (e.g., X-ray diffraction, infrared and solid-state NMR). X-ray diffraction (XRD) is acknowledged to be one of the most sensitive methods to determine the crystallinity of solids. Crystals yield explicit diffraction maxima that arise at specific angles consistent with the lattice interplanar spacings, as predicted by Bragg's law. On the contrary, amorphous materials do not possess long-range order. They often retain additional volume between molecules, as in the liquid state. Amorphous solids normally manifest as a featureless XRD pattern with broad, diffuse halos because of the absence of the long-range order of repeating crystal lattice.

Crystalline forms are preferred in many pharmaceutical applications. Crystalline forms are generally thermodynamically more stable than amorphous forms of the same substance. This thermodynamic stability is preferably reflected in the improved physical stability of the crystalline form. The regular packing of the molecules in the crystalline solid preferably denies the incorporation of chemical impurities. Hence crystalline materials generally possess higher chemical purity than their amorphous counterparts. The packing in the crystalline solid generally constrains the molecules to well defined lattice positions and reduces the molecular mobility that is the prerequisite for chemical reactions. Hence, crystalline solids, with very few notable exceptions, are chemically more stable than amorphous solids of the same molecular composition. Preferably, the crystalline forms of the benzgalantamine gluconate disclosed in the present application possess one or more of the advantageous chemical and/or physical properties disclosed herein.

In other embodiments, the present method leads to synthesis of benzgalantamine, and benzgalantamine gluconate, which can be subsequently prepared in a crystal solid form (e.g., one of Forms A, B, C or D) or a non-crystalline solid form, such as an amorphous form.

As used herein, the term stable may relate to either chemical stability or to polymorph stability. Polymorph stability refers to the likelihood of a polymorph form remaining in its specific crystalline state under suitable storage conditions. For example, a stable polymorph form will maintain at least about 95% by weight, preferably at least about 98% by weight, and more preferably at least about 99% by weight or more of the crystalline form, in other words the form remains unchanged after storage under the indicated conditions for the indicated time. In the context of the present invention, Form A of the benzgalantamine gluconate appears to show good stability for example under conditions of storage at room temperature, and at low water activities, such at or under about 43% RH or of less than 0.12 a_{w}, for multiple months. In some embodiments, Form A shows good chemical stability. In other words, benzgalantamine gluconate as Form A shows low, negligible or no conversion to distinct chemical structures, after storage under the appropriate conditions.

Powder X-ray diffraction (XRPD) measures the diffraction pattern of a crystalline material. Each active pharmaceutical ingredient (API) will produce a specific pattern depending on the structure of its crystal lattice. Each polymorph, pseudopolymorph, polymorph salt, or co-crystalline material will have its own specific pattern. For this reason, XRPD of an API can be carried out in controlled conditions to assess the presence or absence of crystalline material and any form conversions.

XRPD can also be used to determine if any change in crystalline form in the drug product has occurred during e.g. storage or stability studies. The identification of a crystalline form therefore relies on the presence of detectable diffraction peaks for any given crystalline form. In addition, the API peaks must be distinguishable from any crystalline excipient peaks, should a composition be assessed post-formulation. XRPD can also be used as a qualitative and sometime quantitative assessment of the degree of crystallinity of a pure API. A skilled person can assess XRPD patterns and identifying the presence and/or absence of suitable peaks that can be employed to characterize any given crystalline form of an API without undue effort.

In some embodiments, the peaks determined by a XRPD analysis are essentially the same as those presented in the examples. The term “essentially the same” with reference to XRPD means that variabilities in peak positions and relative intensities of the peaks are to be taken into account. For example, a typical precision of the 2-Theta values is in the range of ±0.2° 2-Theta.

As used herein, characteristic XRPD peaks are a subset of the representative peaks from XRPD patterns of a crystalline form of a material that statistically can be proven to differentiate it from the other crystalline forms of that material. Not all crystalline polymorphs of a material necessarily have characteristic peaks.

As used herein, prominent XRPD peaks are typically the strongest low angle, non-overlapping peaks observed in the XRPD pattern. In some embodiments, the “prominent peaks” have preferably a ≥20% relative intensity, preferably ≥30% relative intensity, more preferably ≥40% relative intensity, in a powder X-ray diffraction pattern.

As used herein, representative XRPD Peaks are peaks from XRPD patterns of a crystalline form of a material that statistically show no bias from particle size/shape or preferred orientation during repeated samples and measurements.

As used herein, preferred orientation is phenomena observed in XRPD analyses where due to size/shape of the particles and the pattern collection technique employed it is very difficult or impossible to randomly orient the particles of the material during collection to achieve a pattern with statistically consistent intensities.

With respect to the relative intensities and the prominent peaks of the powder X-ray diffraction patterns mentioned above, the provided values of relative intensity are not intended as limiting for the identification of the prominent or characteristic peaks mentioned. As is known to a skilled person, the relative peak intensities will show some inter-apparatus variability, batch-to-batch variability, as well as variability due to degree of crystallinity, preferred orientation, sample preparation, and as such are provided as an indication and as a qualitative measure only, but not a limiting definition, of the intensities of the peaks in the powder X-ray diffraction patterns.

The term “prominent peak” in the context of defining the present invention is therefore not limited to the respective relative intensities provided above, and any one or more of the respective peaks may be determined as a prominent peak for any given form of benzgalantamine gluconate. Preferably at least 1, 2, 3 or 4 prominent peaks are used to characterize a crystalline form, in other embodiments, at least 5, 6, 7, 8 9 or 10 prominent peaks may be employed. A prominent peak is therefore also not limited to a peak unique to any given crystal form, rather the peak can, optionally in combination with a number of other peaks from the XRPD pattern, be used to identify a crystal form. In the context of the present invention, crystal Forms A-D may share multiple prominent peaks, but also exhibit peaks distinct from one another that can be used to differentiate between any two forms. In some embodiments, the prominent peaks mentioned in the embodiments of the invention may also be characteristic peaks and/or representative peaks.

### Synthesis Process:

The term “base” refers to its established meaning in the art, preferably a chemical compound that can accept protons (H⁺) or donate electron pairs, typically increasing the pH of a solution. Triethylamine (Et₃N) (CAS 121-44-8) is an organic base exhibiting moderate basicity (pka value of 10.76 at 25° C. at 25°) and solubility in water and organic solvents. Its volatility advantageously allows for easy removal from the reaction mixture, which is advantageous in large-scale manufacturing.

The term “halide” refers to its established meaning in the art, preferably a chemical compound that comprising at least one halogen atom (e.g., fluorine, chlorine, bromine, iodine) covalently bound to another atom. Halides can be classified into inorganic halides, such as sodium chloride (NaCl) and magnesium bromide (MgBr₂), and organic halides, such as benzoyl halide (e.g. benzoyl chloride, benzoyl bromide and benzoyl fluoride).

The term “catalyst” refers to its established meaning in the art, preferably a substance that increases the reaction rate of a chemical reaction by lowering the activation energy of the reaction, typically without being consumed by the chemical reaction itself. When used in reference to an esterification reaction such as the reaction of galantamine hydrobromide with a benzoyl halide, the term refers to a substance that increases the reaction rate of the esterification reaction without being consumed itself.

The term “nucleophilic catalyst” refers to a nucleophilic compound that increases the rate of a chemical reaction by temporarily forming a covalent bond with a reactant through a nucleophilic attack. This interaction stabilizes reaction intermediates and lowers the activation energy, facilitating the transformation of the substrate. The catalyst is regenerated at the end of the reaction. Nucleophilic catalysts function by first attacking an electrophilic center, typically forming a transient intermediate that is more reactive than the original substrate. This intermediate then undergoes further transformation, with the catalyst being released at the end of the process. Examples of nucleophilic catalysts include without limitation tertiary amines such as triethylamine, phosphines such as triphenylphosphine and 4-Dimethylaminopyridine (DMAP).

DMAP (CAS 1122-58-3) is a derivative of pyridine and used in organic synthesis as a catalyst due to its high nucleophilicity and ability to stabilize acylated intermediates. It is used for a variety of reactions such as esterifications with anhydrides, the Baylis-Hillman reaction, hydrosilylations, tritylation, the Steglich rearrangement and Staudinger synthesis of p-lactams. Its catalytic efficiency is particularly valuable in esterification and transesterification reactions, where it significantly enhances the rate of ester formation.

Organic solvents are a class of volatile carbon-based chemicals capable of dissolving or dispersing one or more other chemical substances. Common organic solvents are classified as aliphatic hydrocarbons, cyclic hydrocarbons, aromatic hydrocarbons, halogenated hydrocarbons, ketones, amines, esters, alcohols, aldehydes, and ethers. In one embodiment, the preferred organic solvent of the present invention is acetonitrile (ACN).

A distillation refers to its established meaning in the art, preferably a thermal separation process for separation of components or substances from a liquid mixture using the boiling point difference of the mixture and may be carried out below the atmospheric pressure, atmospheric pressure or above the atmospheric pressure. Thereby, the component with the lower boiling point is separated by evaporation and subsequent condensation in a separate vessel. Distillation can be performed in batch or continuous mode. Suitable equipment and an apparatus for performing a distillation is known to a person skilled in the art and comprises at least a vessel wherein the liquid mixture is heated, a heater such as a heating plate or an oil bath or water bath, a condenser in which the heated vapor is condensed such as a Liebig condenser and a receiving vessel in which the condensed liquid is collected.

Azeotropic distillation refers to its established meaning in the art, preferably a specialized separation technique used to break azeotropic mixtures, which are liquid mixtures that maintain a constant composition in both the liquid and vapor phases during boiling and therefore cannot be separated into pure components by simple distillation. Since conventional distillation relies on differences in volatility, azeotropic distillation introduces an additional component as an entrainer or modifies pressure conditions to alter the phase behavior, thereby allowing separation of the components of the azeotropic mixture. There are two main approaches to azeotropic distillation. In heterogeneous azeotropic distillation, an entrainer is added that induces phase separation in the distillate, forming two immiscible liquid layers. One layer is rich in the desired product, while the other contains the entrainer and the unwanted component, allowing for easy separation by decantation. A typical example is the dehydration of ethanol using benzene, toluene, or cyclohexane, which aid at removing water by forming a low-boiling ternary azeotrope that distills off first. In contrast, homogeneous azeotropic distillation involves the addition of an entrainer that modifies the relative volatilities of the components without causing phase separation. The entrainer forms a new azeotropic composition, allowing selective removal of one component and breaking the original azeotrope. An example is the use of acetone to separate water from methanol-water mixtures. Additionally, pressure-swing distillation exploits the fact that some azeotropes shift their composition or disappear entirely at different pressures. By performing distillation at varying pressures, separation becomes possible. A well-known example of this method is the separation of hydrochloric acid from water, where different azeotropic compositions exist at low and high pressures, allowing for separation through consecutive pressure changes.

The term “impurity” refers to any additional, usually unintended, compound present in the reaction product, intermediate, or raw material. Impurities can originate from various sources, including material related impurities such as impurities present in starting materials, such as epi-galantamine, lycoramine, O-desmethyl-galantamine, N-oxide of galantamine and N-desmethylgalantamine present in galantamine hydrobromide, other reagents and solvents, and process-related impurities which arise from side reactions during the manufacturing process such as the process related impurities listed in table 17.

### Analytical Techniques and Terms:

As used herein, DSC (Differential Scanning Calorimetry) is a thermodynamic technique for assessment of the heat energy changes occurring in a sample undergoing a physical or chemical change with a controlled change in temperature.

As used herein, FTIR (Fourier Transform Infrared Spectroscopy) is a simple and reliable technique widely used in the pharmaceutical industry for identification and characterization of materials. The Infrared spectrum can be used for identification of materials because the functional groups of the material give rise to characteristic vibrational bands in terms of both intensity and frequency.

As used herein, NMR (Nuclear Magnetic Resonance) is a technique in which the sample is placed in a strong magnetic field and the NMR active nuclei absorb electromagnetic radiation at a frequency characteristic of the sample. The most common types are proton (1H) and carbon (13C) NMR.

As used herein, TGA (Thermo-Gravimetric Analysis) is a thermogravimetric technique to monitor the changes in the mass of a sample as a function of time and/or temperature.

As used herein, XRPD (X-Ray Powder Diffraction) is a technique for identification and characterization of crystalline materials to ensure the proper phase or polymorph is present. The technique can be used to measure the sample in either a transmission or reflectance mode for analysis.

As used herein, RH (relative humidity) refers to the ratio of the partial pressure of water vapor to the equilibrium vapor pressure of water at a given temperature. In order to determine RH, a hygrometer may be used for measuring the humidity of air. Relative humidity is expressed as a percentage; a higher percentage means that the air-water mixture is more humid.

### Compositions and Application

The term “active ingredient” or “API” herein refers to the relevant drug molecule (e.g. benzgalantamine) as well as its pharmaceutically acceptable and therapeutically active salts, esters, amides, prodrugs, metabolites, enantiomers, polymorphs, analogs, etc. that induce a desired pharmacological or physiological effect. Terms like “active”, “active agent”, “active substance” may be used synonymously for “active ingredient”. In the context of the present application, benzgalantamine is considered as an API, although not active itself prior to enzymatic cleavage, it is the pro-drug molecule to be prepared and/or formulated.

“Administration” or “treatment,” as it applies to an animal, human, experimental subject, cell, tissue, organ, or biological fluid, refers to contact of a pharmaceutical, therapeutic, diagnostic agent, compound, or composition to the animal, human, subject, cell, tissue, organ, or biological fluid. “Administration” and “treatment” can refer, e.g., to therapeutic, placebo, pharmacokinetic, diagnostic, research, and experimental methods. The term “subject” includes both human and veterinary subjects. “Treatment,” as it applies to a human, veterinary, or research subject, refers to therapeutic treatment, prophylactic or preventative measures, to research and diagnostic applications.

The invention encompasses the administration of an effective amount of chemical substance as described herein to a subject or patient in need thereof. “Effective amount” or “therapeutically effective amount” means an amount sufficient to elicit an appreciable biological response such as ameliorate a symptom or sign of a disorder or physiological condition when administered to a subject or patient. An effective amount for a particular patient or veterinary subject may vary depending on factors such as the condition being treated and the overall health and age of the patient. An effective amount can be the maximal dose or dosing protocol that avoids significant side effects or toxic effects. “Effective amount” also relates to an amount of the prodrug substance or pharmaceutical composition thereof, sufficient to allow or facilitate appreciable biological response such as the amelioration and of a symptom or sign of a disorder, condition, or pathological state.

Suitable routes of administration may include, without limitation, oral, rectal, transmucosal or intestinal administration or intramuscular, subcutaneous, intramedullary, intrathecal, direct intraventricular, intravenous, intravitreal, intraperitoneal, intranasal, or intraocular injections. The preferred routes of administration are transmucosal or oral.

The pharmaceutical composition may include one or more pharmaceutically acceptable carriers, or excipients. The term “excipient” means a pharmacologically inactive component such as a diluent, disintegrant, carrier, and the like, of a pharmaceutical product. The excipients that are useful in preparing a pharmaceutical composition are generally safe, non-toxic and are acceptable for veterinary as well as human pharmaceutical use. Reference to an excipient includes both one excipient and more than one excipient. The excipients are described herein in some embodiments according to “wt %”, or “percentage by weight”.

Pharmaceutical compositions of the present invention may be manufactured by processes well known in the art, e.g., by means of conventional mixing, dissolving, granulating, dragee-making, levigating (micronizing), emulsifying, encapsulating, entrapping, dissolving or lyophilizing processes. Pharmaceutical compositions for use in accordance with the present invention may be formulated in conventional manner using one or more physiologically acceptable carriers including excipients and auxiliaries that facilitate processing of crystals of the present invention into preparations that can be used pharmaceutically. Proper formulation is dependent upon the route of administration chosen.

For injection or transmucosal administration, a product of the present invention or a pharmaceutical composition thereof may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, a product of the present invention or a pharmaceutical composition thereof can be formulated by combining the product of the present invention with pharmaceutically acceptable carriers well known in the art. Such carriers allow a product of the present invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, starch and other materials. If desired, disintegrating agents may be added.

For administration by inhalation, a product of the present invention or a pharmaceutical composition thereof is conveniently delivered in the form of an aerosol spray using a pressurized pack or a nebulizer and a suitable propellant, e.g., without limitation, dichlorodifluoromethane, trichlorofluoromethane, dichlorotetrafluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of a crystal of the present invention or a pharmaceutical composition thereof, and a suitable powder base such as lactose or starch.

A product of the present invention or a pharmaceutical composition thereof may also be formulated for parenteral administration, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulating materials such as suspending, stabilizing, and/or dispersing agents.

Pharmaceutical compositions for parenteral administration include aqueous solutions of a water-soluble form of a product of the present invention or pharmaceutical composition thereof. Additionally, suspensions of a product of the present invention or pharmaceutical compositions thereof may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances that increase the viscosity of the suspension, such as sodium carboxymethyl cellulose, sorbitol, or dextran. Optionally, the suspension may also contain suitable stabilizers and/or agents that increase the solubility of a product of the present invention or a pharmaceutical composition thereof to allow for the preparation of highly concentrated solutions. Alternatively, the active ingredient may be in powder form for constitution with a suitable vehicle, e.g., sterile, pyrogen-free water, before use.

A product of the present invention or a pharmaceutical composition thereof may also be formulated in rectal compositions such as suppositories or retention enemas, using, e.g., conventional suppository bases such as cocoa butter or other glycerides.

A preferred mode of administration according to the present invention is transmucosal administration. The term “transmucosal administration” relates to the entering of a pharmaceutical agent through, or across, a mucous membrane. The transmucosal routes of administration of the present invention are preferably intranasal, buccal and/or sublingual.

Nasal or intranasal administration relates to any form of application to the nasal cavity. The nasal cavity is covered by a thin mucosa which is well vascularized. Therefore, a drug molecule can be transferred quickly across the single epithelial cell layer without first-pass hepatic and intestinal metabolism. Intranasal administration is therefore used as an alternative to oral administration of for example tablets and capsules, which lead to extensive degradation in the gut and/or liver.

Buccal administration relates to any form of application that leads to absorption across the buccal mucosa, preferably pertaining to adsorption at the inside of the cheek, the surface of a tooth, or the gum beside the cheek. Sublingual administration refers to administration under the tongue, whereby the chemical comes in contact with the mucous membrane beneath the tongue and diffuses through it.

Pharmaceutical compositions suitable for buccal and/or sub-lingual administration may comprise additional pharmaceutically acceptable carriers or excipients. The active agent can be physically compounded with materials of some or all of classes of ingredients that function as pH controls, preservative agents, viscosity control agents, absorption enhancers, stabilizing agents, solvents, mucoadhesives, flavouring agents and carrier vehicles. Such agents may be present in either solid or liquid forms of the pharmaceutical composition.

Determination of a therapeutically effective amount and suitable mode of administration is within the capability of those skilled in the art, especially in light of the detailed disclosure provided herein. The amount of a composition administered will, of course, be dependent on the subject being treated, the severity of the affliction, the manner of administration, the judgment of the prescribing physician, etc.

### FIGURES

The invention is demonstrated by way of the example through the figures disclosed herein. The figures provided represent particular, non-limiting embodiments and are not intended to limit the scope of the invention.

FIG. 1: Molecular Structure of Benzgalantamine Gluconate labelled with carbon numbering.

FIG. 2: ¹H ¹³C HSQC Spectrum of Benzgalantamine Gluconate.

FIG. 3: ¹H-¹H COSY Spectrum of Benzgalantamine Gluconate.

FIG. 4: ¹H-¹³C HMBC Spectrum of Benzgalantamine Gluconate.

FIG. 5: Schematic Diagram of Correlation of Spectrum of Benzgalantamine Gluconate by COSY and HMBC.

FIG. 6: IR Spectrum of Benzgalantamine Gluconate.

FIG. 7: Ultraviolet-visible Absorption Spectrum of Benzgalantamine Gluconate.

FIG. 8: Determination of enantiomeric purity by HPLC for Benzgalantamine Gluconate.

FIG. 9: XRPD Pattern of Benzgalantamine Gluconate Form A Anhydrate.

FIG. 10: Typical DSC Thermogram, Benzgalantamine Gluconate.

FIG. 11: XRPD of Benzgalantamine gluconate after azeotropic distillation (dry cake). STD: standard.

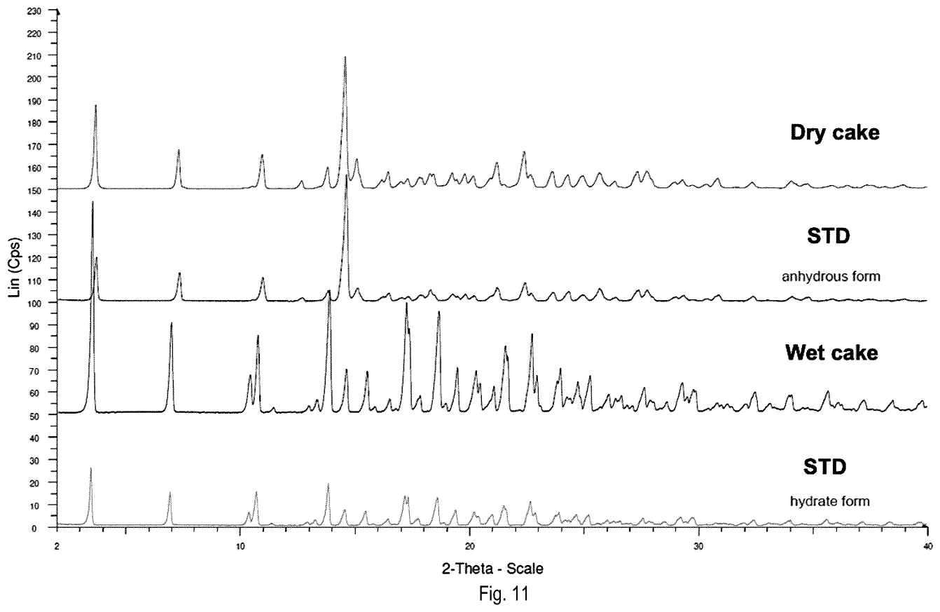

### EXAMPLES

The invention is demonstrated through the examples disclosed herein. The examples provided represent particular embodiments and are not intended to limit the scope of the invention. The examples are to be considered as providing a non-limiting illustration and technical support for carrying out the invention.

### I. Synthesis Process

### 1. Abbreviations:

SPT2455SM1: Galantamine hydrobromide (Galantamine HBr); SPT2455M1: Benzgalantamine; GLN-1062 gluconate: Benzgalantamine gluconate; BzCl: Benzoyl chloride; Et₃N: Triethylamine; DMAP: 4-Dimethylaminopyridine; ACN: Acetonitrile; IPAc: Isopropyl acetate; MEK: Methyl ethyl ketone

2. Description of the Manufacturing Process and in-Process Control:

Benzgalantamine gluconate is synthesized in a two-step process. The synthesis starts with the esterification of galantamine hydrobromide using benzoyl chloride to produce galantamine benzoic ester (Benzgalantamine, or Galantamine benzoate) (Step 1, Scheme 1, Table 1). Benzgalantamine is then treated with D-(+)-gluconolactone to form benzgalantamine gluconate (Step 2, Scheme 2, Table 2).

The reaction temperatures, times, and quantities indicated may be varied within the proven acceptable ranges (PARs) to improve process efficiency without adversely affecting synthesis product quality.

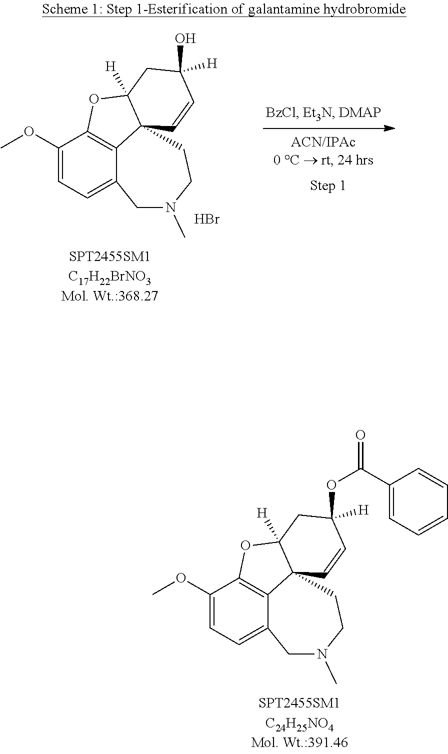 [SMILES: COc1ccc2c3c1O[C@H]1C[C@@H](O)C=C[C@@]31CCN(C)C2 ; Br ; COc1ccc2c3c1O[C@H]1C[C@@H](OC(=O)c4ccccc4)C=C[C@@]31CCN(C)C2]

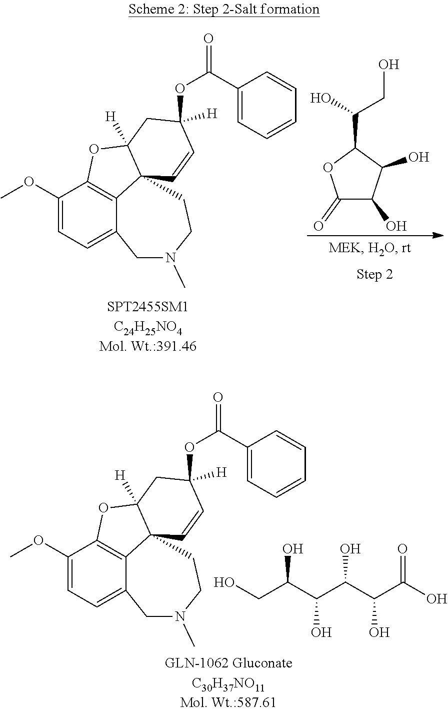

A detailed description of the manufacturing procedure and in-process control (IPCs) is provided in Tables 1 and 2.

TABLE 1
Step 1- Esterification of galantamine hydrobromide

| Step | Process step description | In-process control (IPC) |
|---|---|---|
| IPC1 | In-process control (IPC) | Water content of acetonitrile, not more |
| | | than 0.04% |
| 1.1 | Charge galantamine hydrobromide (1 | |
| | equiv.) and acetonitrile (8.0 vol) at | |
| | 20° C. ± 10° C. | |
| 1.2 | Add triethylamine (3-5 equiv.) at 20° C. ± | |
| | 10° C. | |
| 1.3 | Add DMAP (0.1 equiv.) in acetonitrile | |
| | (0.3 vol) at 20° C. ± 10° C. and stir at | |
| | 20° C. ± 5° C. for not less than 1 h | |
| 1.4 | Cool reaction mass/suspension to 0° C. ± | |
| | 10° C. | |
| 1.5 | Add benzoyl chloride (0.6 equiv.) in | Addition period: Not less than 60 min |
| | isopropyl acetate (1 vol.) at 0 ± 10° C. | Overall target amount benzoyl chloride: |
| | over not less than 60 min | 1.2 equiv |
| | | Normal operating range (NOR): |
| | | 1.15 equiv to 1.25 equiv benzoyl chloride |
| | | Proven acceptable range (PAR): |
| | | 1.1 equiv to 1.7 equiv benzoyl chloride |
| | | Target addition temperature: 0° C. |
| | | Addition temperature range: −5° C. to 5° C. |
| | | PAR for addition temperature: −10° C. to |
| | | 10° C. |
| 1.6 | Stir at 0° C. ± 10° C. for not less than 1 h | Temperature range: −10° C. to 10° C. |
| 1.7 | Heat to 20° C. ± 10° C. over not less | |
| | than 1 h, Stir at 20° C. ± 10° C. for not | |
| | less than 4 h | |
| 1.8 | Cool to 0° C. ± 10° C. | |
| 1.9 | Add benzoyl chloride (0.60 equiv.) in | Refer step 1.5. |
| | isopropyl acetate (1 vol.) at 0 ± 10° C. | Alternatively, add 1.2 equivalents benzoyl |
| | over not less than 60 min | chloride in a single step at 1.5, and skip |
| | | 1.9-1.10. |
| 1.10 | Stir at 0° C. ± 3° C. for not less than 1 h, | |
| | Heat to 20° C. ± 3° C. over not less than | |
| | 1 h, Stir at 20° C. ± 3° C. for not less | |
| | than 2 h. | |
| IPC2 | In-process control (IPC) | Residual galantamine HBr content: not |
| | | more than 3% |
| 1.11 | Add isopropyl acetate (8.0 vol) and stir | |
| | at 20° C. ± 10° C. | |
| 1.12 | Filter and wash filter cake with | |
| | isopropyl acetate | |
| 1.13 | Concentrate (vacuum distill), repeat | |
| | wash with isopropyl acetate, cool to | |
| | 20° C. ± 10° C. | |
| 1.14 | Add water (2-3 vol) and stir at 20° C. ± | |
| | 10° C. for 4 h or more. | |
| 1.15 | Add sodium bicarbonate (2.5 vol) and | |
| | extract the organic phase; stir and let | |
| | settle for phases to separate. | |
| 1.16 | Extract the organic phase with sodium | |
| | bicarbonate solution, potentially | |
| | repeat. | |
| 1.17 | Extract the organic phase with sodium | |
| | chloride solution. | |
| 1.18 | Rinse with isopropyl acetate (at least 2 | |
| | vol), add sodium sulfate to organic | |
| | phase, filter and wash cake with | |
| | isopropyl acetate, concentrate by | |
| | vacuum distill, cool to 30° C. ± 5° C. | |
| 1.19 | Add n-heptane (5 vol) and stir at 30° C. ± | not less than 20° C. |
| | 5° C. Repeat n-heptane and vacuum | |
| | distill to remove residual isopropyl | |
| | acetate. | |
| IPC3 | In-process control (IPC) | Residual isopropyl acetate content: not |
| | | more than 2%. |
| | | If IPC3 fails, concentrate by vacuum distill |
| | | and repeat n-heptane stirring. Retest |
| | | IPC3 until it passes. |
| 1.20 | Cool and stir at 20° C. ± 5° C. | |
| 1.21 | Filter and wash cake with 99% n- | |
| | heptane and dry under vacuum | |
| IPC4 | In-process control (IPC) | Loss on drying of benzgalantamine: |
| | | continue drying until IPC meets criteria |
| | | not more than 1.0%, continue with IPC5 |
| IPC5 | In-process control (IPC) | Appearance: white to pale brown solid |
| | | Identification of benzgalantamine |
| | | ¹H-NMR: conforms to reference spectrum |
| | | Purity: NLT 97.0% |
| 1.22 | Package dry product | |

TABLE 2
Step 2 - Salt formation

| Step | Process description | In process control (IPC) |
|---|---|---|
| 2.1 | Charge methyl ethyl ketone (MEK, 6 | |
| | vol) and dissolve benzgalantamine (1 | |
| | eqiv) in MEK | |
| 2.2 | Stir mixture at 15° C. to 25° C. to give a | |
| | clear solution | |
| 2.3 | Filter and rinse with MEK (7 vol) | |
| 2.4 | Add D-(+)-gluconolactone ([1 eqiv] in | Overall MEK/H₂O ratio (vol./vol.; steps |
| | water [1 vol];¹ solution stirred at 15° C. | 2.1 to 2.4) between 10/1 to 15/1 |
| | to 25° C. to give clear solution) and stir | |
| | for not less than 5 h. | |
| 2.6 | Add benzgalantamine gluconate seed, | |
| | 0.4% in MEK stirred at 15° C. to 25° C. | |
| | Precipitation will occur. If precipitation | |
| | is not observed, add additional seed | |
| | (0.4% in MEK) and stir until | |
| | precipitation occurs | |
| 2.7 | When solid is observed, stir mass at | |
| | 15° C. to 25° C. for not less than 12 h | |
| 2.8 | Concentrate the slurry to ~10 vol at | |
| | not more than 40° C. | |
| 2.9 | Solvent swap with MEK; concentrate | |
| | to ~10 vol at not more than 40° C. | |
| IPC6 | In-process control (IPC) | Water content (KF), not more than 4% |
| | | If IPC6 fails, repeat solvent swap with |
| | | MEK until IPC meets criteria. |
| 2.10 | Stir at 15° C. to 25° C. for at least 2 h | |
| 2.11 | Filter and wash cake with MEK | |
| 2.12 | Dry filter cake in a vacuum | |
| IPC7 | In-process control (IPC) | Residual MEK (¹H-NMR): not detected |
| | | Residual water content (KF): not more |
| | | than 9% |
| | | If IPC7 (residual MEK and water |
| | | contents) do not meet criteria, continue |
| | | drying. |
| 2.13 | Dry wet cake under vacuum | |
| IPC8 | In-process control (IPC) | Verify anhydrate form (Form A) (XRPD) |
| | | Residual MEK not more than 4000 ppm |
| IPC9 | In-process control (IPC) | Determination of impurities (see section |
| | | 1.3. and section III. - Impurity control |
| | | below) |

¹Optionally dissolve D-(+)-gluconolactone (1 equiv.) In water (0.5 vol.), charge through a filter, add water to rinse the filter.

### 3. In-Process Controls

The synthesis of benzgalantamine gluconate is controlled by in-process controls (IPCs). The IPC attributes, acceptance criteria and analytical procedures are summarized in Table 3.

TABLE 3
In-process controls (IPCs)

| | | | |
|---|---|---|---|
| IPC | In-Process Control Attribute | Acceptance Criteria | Test Method |

Step 1

| | | | |
|---|---|---|---|
| IPC1 | Residual water content of | ≤0.04% | According to USP |
| | acetonitrile | | <921> (Karl-Fisher |
| | | | titration) |
| IPC2 | Residual galantamine HBr | ≤3% | HPLC |
| | content | | |
| IPC3 | Residual isopropyl acetate | ≤2% | According to USP |
| | content | | <761> (¹H NMR) |
| IPC4 | Loss on drying | ≤1.0% | According to USP |
| | | | <891> (TGA) |
| IPC5 | Appearance | White to pale brown | Visual examination |
| | | solid | |
| | Identification, benzgalantamine | Conforms to | According to USP |
| | | reference standard | <761> (¹H NMR) |
| | Purity, benzgalantamine | ≥97.0% | HPLC |

Step 2

| | | | |
|---|---|---|---|
| IPC6 | Water content in MEK mother | ≤4% | According to USP |
| | liquor | | <921> (Karl-Fisher |
| | | | titration) |
| IPC7 | Residual methyl ethyl ketone | Not detected | According to USP |
| | (MEK) in filter cake | | <761> (¹H NMR) |
| | Water content of | ≤9% | According to USP |
| | benzgalantamine gluconate | | <921> (Karl-Fisher |
| | | | titration) |
| IPC8 | Residual MEK in | ≤4000 ppm | GC |
| | benzgalantamine gluconate | | |
| | Anhydrate form | Conforms anhydrate | According to USP |
| | | form | <941> (XRPD) |
| ICP9 | Impurities (see also section I.3. | | HPLC |
| | and section III. - Impurity control | | |
| | below) | | |
| | Any unspecified impurity | ≤0.10% | |
| | Galantamine HBr | ≤0.15% | |
| | epi-Benzgalantamine | ≤0.15% | |
| | nor-Benzgalantamine | ≤0.15% | |
| | N-oxide of Benzgalantamine | ≤0.15% | |
| | Total impurities | ≤1.0% | |

TABLE 4
HPLC Gradient conditions
Column: Luna Phenyl-Hexyl, 4.6 mm*150 mm, 5 μm or equivalent

| Time | Mobile phase A (%) | Mobile phase B (%) | |
|---|---|---|---|
| (min) | 0.1% HCOOH in H₂O | 0.1% HCOOH in ACN | Flow Rate |
| 0 | 90 | 10 | 1.5 mL/min |
| 9 | 5 | 95 | 1.5 mL/min |
| 11 | 5 | 95 | 1.5 mL/min |
| 11.1 | 90 | 10 | 1.5 mL/min |
| 15 | 90 | 10 | 1.5 mL/min |

TABLE 5
HPLC conditions and retention times.

| Injection volume | 5 | μL |
|---|---|---|
| Detection | UV, 230 nm | |
| Column Temp | 40° | C. |
| Sample compartment temperature | 5° | C. |
| Sample solvent | ACN/water, v/v = 1/1 | |
| Post time | 15.0 | min |

| Components | RT (min) | RRT |
|---|---|---|
| Dimethylaminopyridine (DMAP) | 0.90 | 0.21 |
| Nor-Galantamine | 1.04 | 0.24 |
| Galantamine | 1.24 | 0.29 |
| Epi-Galantamine | 1.74 | 0.40 |
| Narwedine | 2.38 | 0.55 |
| Nor-Benzgalantamine | 4.23 | 0.98 |
| Benzgalantamine | 4.32 | 1.00 |
| N-oxide of benzgalantamine | 4.49 | 1.04 |
| Epi-Benzgalantamine | 4.62 | 1.07 |
| Benzoic acid | 4.87 | 1.13 |
| Benzoyl chloride | 8.16 | 1.89 |

### Method for HPLC of IPC9:

The conditions for the HPLC method of IPC9 are disclosed in the following tables 6 to 8.

TABLE 6
HPLC Gradient Conditions
Column: Macherey-Nagel Nucleodur C18 Gravity,
4.6 mm*150 mm, 3 μm or equivalent

| Time | Mobile phase A (%) | Mobile phase B (%) | |
|---|---|---|---|
| (min) | 0.1% (v/v) TFA in H₂O | 0.1% (v/v) TFA in ACN | Flow Rate |
| 0 | 90 | 10 | 1.5 mL/min |
| 45 | 25 | 75 | 1.5 mL/min |
| 45.1 | 90 | 10 | 1.5 mL/min |
| 50 | 90 | 10 | 1.5 mL/min |

TABLE 7
HPLC conditions.

| Injection volume | 10 | μL |
|---|---|---|
| Detection | UV, 230 nm | |
| Column Temp | 40° | C. |
| Sample compartment temperature | 10° | C. |
| Sample solvent | ACN/water (v/v = 1/4) | |
| Post time | 50.0 | min |

TABLE 8
Retention time.

| Components | RT (min) | RRT |
|---|---|---|
| D-(+)-Gluconolactone | 0.94 | 0.05 |
| Nor-Galantamine | 2.62 | 0.15 |
| Galantamine | 3.18 | 0.19 |
| Epi-Galantamine | 4.48 | 0.26 |
| Narwedine | 5.54 | 0.33 |
| Nor benzgalantamine | 16.88 | 0.99 |
| Benzgalantamine Gluconate | 17.02 | 1.00 |
| N-oxide benzgalantamine | 18.01 | 1.06 |
| Epi benzgalantamine | 19.63 | 1.15 |

### II. Characterization Studies of Benzgalantamine Gluconate Produced by the Synthesis Process Described in Section I.:

### 1. Elemental Analysis

The elemental carbon, hydrogen, nitrogen, and oxygen composition of batch 71668AA001 corrected for water content, was determined and compared to the theoretical elemental composition.

The measured elemental composition was consistent with the theoretical elemental composition of benzgalantamine gluconate, C₃₀H₃₇NO₁₁ (Table 9).

TABLE 9
Results of elemental analysis of benzgalantamine
gluconate (batch 71668AA001)
Resultsₙ₌₂ (%)

| | C | H | N | O |
|---|---|---|---|---|
| Average | 61.41 | 6.54 | 2.46 | 29.53 |
| Theoretical | 61.16 | 6.36 | 2.38 | 30.11 |
| value | | | | |

### 2. Mass Spectrometry

The electrospray ionization mass spectrogram of benzgalantamine gluconate was obtained using an LTQ-XL mass spectrometer equipped with a linear ion trap mass analyzer.

The sample yielded a protonated ion [M+H]⁺ at m/z=392.2. The monoisotopic mass (M) was 391.2 Da, which conforms to the expected monoisotopic mass of benzgalantamine gluconate (391.18 Da as free form). The major m/z values (ESI⁺/MS/MS) of three batches are provided in Table 10. All fragment ions conform to the proposed structure. The errors for all measurements are within ±0.5 amu.

TABLE 10
Spectral Assignment of ESI⁺/MS/MS spectrum
of Benzgalantamine Gluconate (as free form).

| m/z | | | Assigment |
|---|---|---|---|
| Batch | Batch | Batch | (M = |
| 71668AA001 | 71668AA002 | 71668AA003 | C₂₄N₂₈N₂O₃) |
| 392.2 | 392.2 | 392.2 | [M + H]⁺ |
| 270.2 | 270.2 | 270.2 | [M + |
| | | | H—C₇H₆O₂]⁺ |
| 213.1 | 213.1 | 213.1 | [M + |
| | | | H—C₁₀H₁₃NO₂]⁺ |

### 3. Nuclear Magnetic Resonance (NMR)

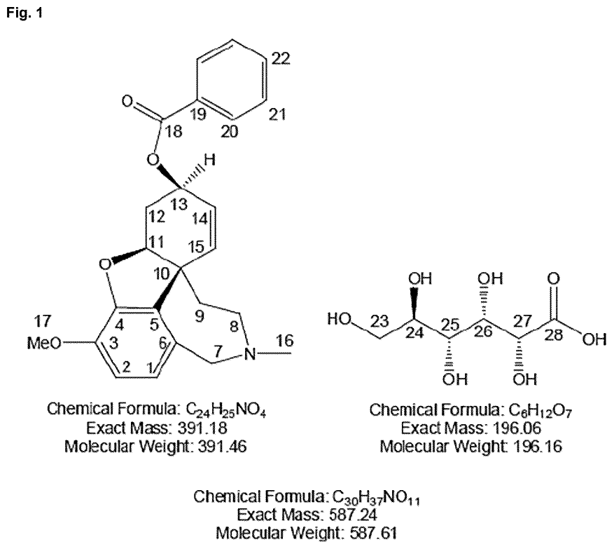

The structure of benzgalantamine gluconate labeled with carbon numbering is shown in FIG. 1.

The ¹H NMR and ¹³C NMR spectra of benzgalantamine gluconate in D₂O containing 0.75% wt. % 3-(trimethylsilyl)propionic-2,2,3,3-d₄ acid, sodium salt were acquired using a Bruker 400 MHz Ultrashield™ NMR spectrometer.

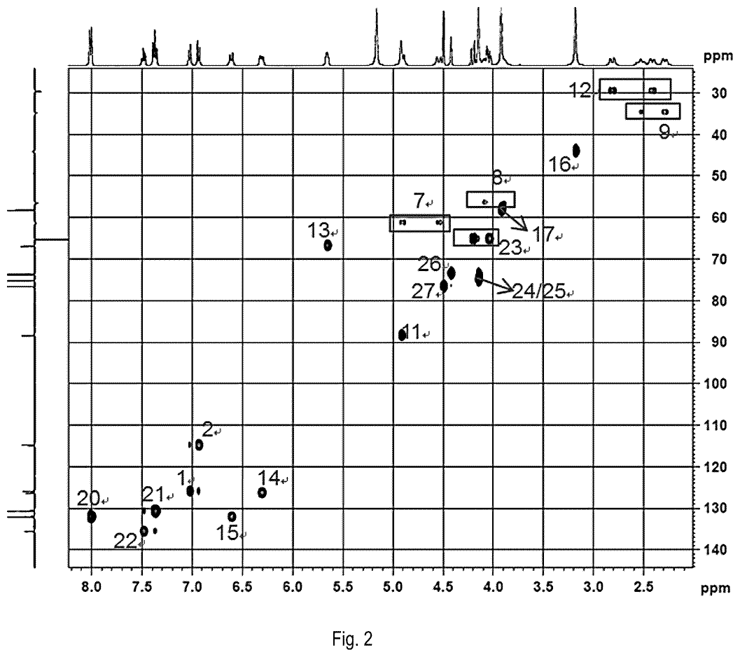

The structure of benzgalantamine gluconate was also characterized by 2D NMR in which the 2D ¹H-¹³C heteronuclear single-quantum coherence (HSQC) spectrum is correlated with the proton directly bonded carbon. The carbon-proton correlation spectrum is provided in FIG. 2.

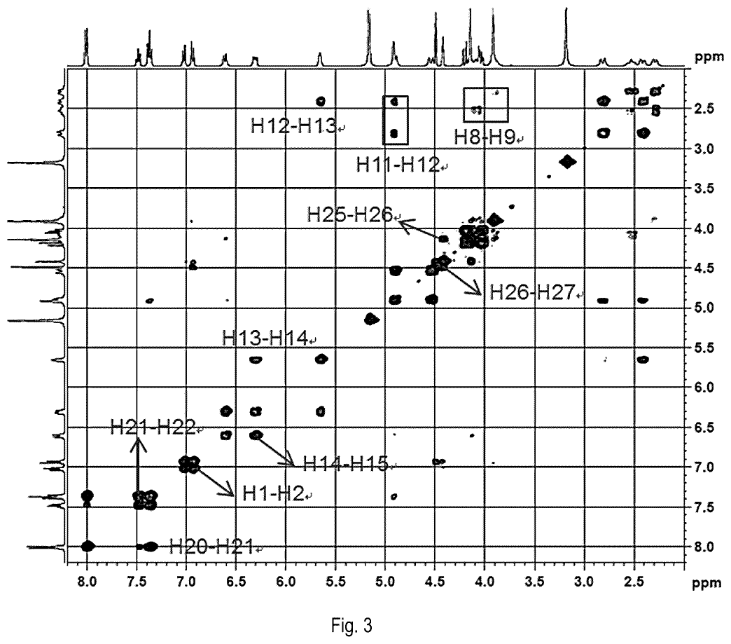

The 2D ¹H-¹H chemical shift correlated spectroscopy (COSY) spectrum shown in FIG. 3 correlated the adjacent protons via two or three bonds which allowed the assignment of five sets of fragments.

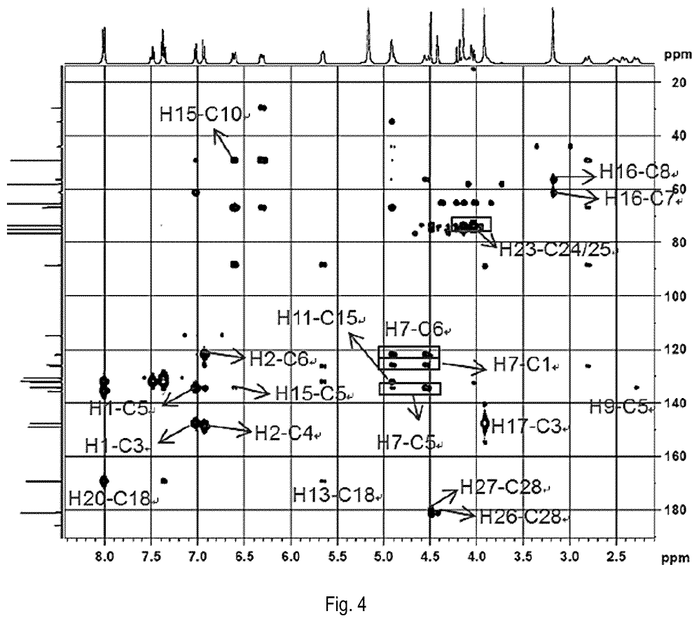

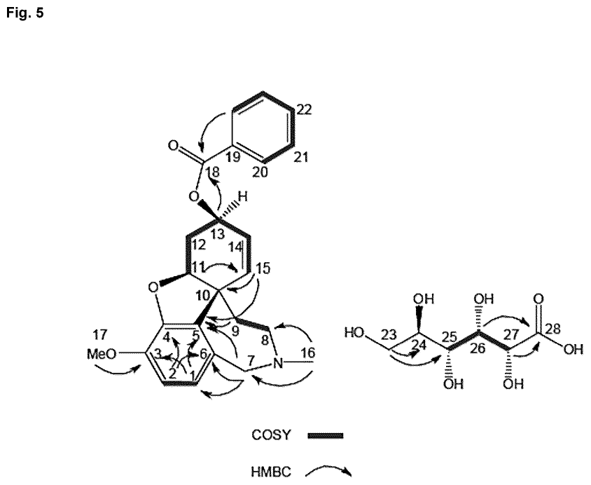

The 2D ¹H-¹³C heteronuclear multiple-bond correlation (HMBC) spectrum detected the connectivity between protons and carbons through two to four bonds. The HMBC spectrum shown in FIG. 4 and the correlation between carbon-proton by COSY and HMBC are provided in FIG. 5.

The NMR spectral assignments are based on the chemical shifts, splitting patterns (multiplicity) and integrations of hydrogen atoms in ¹H NMR spectrum as well as the chemical shifts in ¹³C-NMR, as detailed in Table 11. The spectral assignments conformed to the proposed structure of benzgalantamine gluconate shown in FIG. 5.

TABLE 11
Spectral assignments for ¹³C-NMR and ¹H-NMR Spectra of
Benzgalantamine Gluconate (in D2O), Batch 71668AA001.

| Atom position | ¹³C Chemical | |
|---|---|---|
| Numbering ^{a} | Shift (ppm) | 1H-NMR ^{b} |
| 1 | 126.0 | 6.94 (1H, d, J = 8.4) |
| 2 | 114.9 | 7.03 (1H, d, J = 8.4) |
| 3 | 147.8 | — |
| 4 | 149.0 | — |
| 5 | 134.4 | — |
| 6 | 122.1 | — |
| 7 | 61.4 | 4.54 (1H, d, J = 14.8, |
| | | 4.91 (1H, m)) |
| 8 | 56.5 | 3.89 (1H, m) |
| | | 2.29 (1H, d, J = 12.0) |
| 9 | 34.8 | 2.53 (1H, t, J = 13.4) |
| 10 | 49.3 | — |
| 11 | 88.5 | 4.92 (1H, s) |
| 12 | 29.7 | 2.42 (1H, d, J = 13.2) |
| | | 2.82 (1H, d, J = 14.8) |
| 13 | 66.9 | ac5.66 (1H, s) |
| 14 | 126.4 | 6.31 (1H, dd, J = 10.0, 5.2) |
| 15 | 132.1 | 6.61 (1H, d, J = 10.4) |
| 16 | 44.1 | 3.18 (3H, brs) |
| 17 | 58.2 | 3.92 (3H, s) |
| 18 | 169.4 | — |
| 19 | 132.3 | — |
| 20 | 132.1 | 8.01 (2H, d, J = 7.6) |
| 21 | 130.7 | 7.37 (2H, t, J = 7.6) |
| 22 | 135.6 | 7.49 (1H, t, J = 7.4) |
| 23 | 65.3 | 4.04 (1H, m), |
| | | 4.21 (1H, m) |
| 24 | 73.8 | 4.15 (1H, m) |
| 25 | 75.2 | 4.15 (1H, m) |
| 26 | 73.6 | 4.42 (1H, m) |
| 27 | 76.7 | 4.50 (1H, d, J = 3.6) |
| 28 | 181.2 | — |

^{a} atom numbering is labelled in FIG. 5.
^{b} s = singlet, dd = doublet of doublet; m = multiplet

### 4. Infrared Spectrophotometry (IR Spectrophotometry)

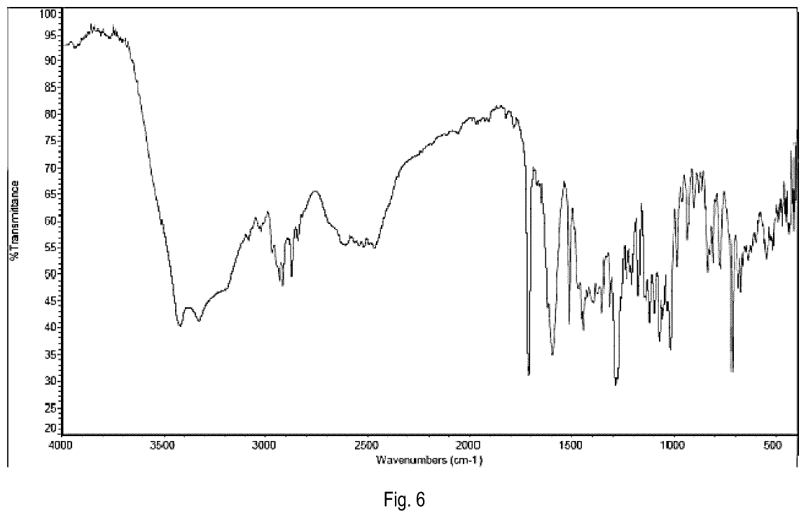

The IR spectrum of benzgalantamine gluconate was acquired using a NICOLE (model: iS10) FT-IR using potassium bromide and a diffuse reflectance accessory. The spectrum from 4000 cm⁻¹ to 400 cm⁻¹ is provided in FIG. 6; assignments for the characteristic bands are provided in Table 12.

TABLE 12
Characteristic Absorption Bands and Their Assignments, IR
Spectrum of Benzgalantamine Gluconate, Batch 71668AA001.

| Wavenumber range (cm⁻¹) | Intensity | Assignment |
|---|---|---|
| 3500-3250 | Strong | O—H stretch |
| 3100-2400 | Broad, moderate | COO—H stretch |
| 3100-3000 | Moderate | Aromatic C—H stretch |
| 2980-2820 | Moderate | Aliphatic C—H stretch |
| 1720-1700 | Strong | C═O stretching |
| 1630-1440 | Strong | Aromatic C═C, |
| | | C═N stretch |
| 1360-1050 | Strong | C—N stretch |
| 1300-1000 | Strong | C—O stretch |

### 5. Ultraviolet-Visible Spectrophotometry

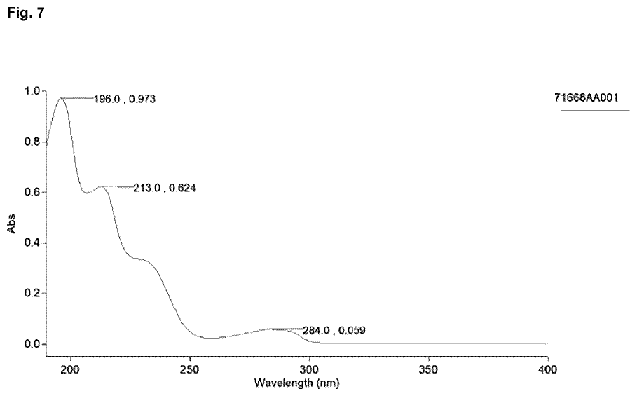

The ultraviolet absorption spectrum of benzgalantamine gluconate in water (0.008 mg/mL) was obtained using an Agilent Cary 60 ultraviolet-visible spectrophotometer; the resulting spectrum is provided in FIG. 7.

### 6. Isomerism/Stereochemistry

Galantamine hydrobromide provides the core skeleton of benzgalantamine gluconate, the chirality centers and stereoisomers of which are established and well controlled during the synthesis of galantamine HBr. The stereoisomer content of epi-galantamine is controlled at not more than 0.20%. The corresponding impurity, epi-benzgalantamine, originating from epi-galantamine during the manufacture of benzgalantamine gluconate, can be removed completely and is controlled at not more than 0.15%.

The enantiomeric purity of (+)-benzgalantamine gluconate (batches 711668AA004, 71714AA002, and 71714AA003) was verified using normal phase HPLC.

### HPLC Conditions:

Column: Daicel Chiracel OD, 250 mm (L)×4.6 mm(ID), 10 m particle size, Eluent: n-Hexane:EtOH:DEA=50:50:0.2, isocratic, Flow rate: 0.5 mL/min, Injection volume: 10 μL, Column temperature: Ambient, Sample temperature: Ambient, Sample run time: 30 minutes, Detector: UV, 230 nm, Sample solvent: Methanol

### Procedure:

### I. Blank Solution

### Sample Solvent

### II. 1% Standard Solution

Accurately weigh about 12.5 mg of Benzgalantamine racemic mixture standard into a 25-mL volumetric flask and dilute to volume with sample solvent and mix well. Pipette 1 mL of standard solution into a 50-mL volumetric flask, add sample solvent to the mark and mix well.

### III. System Suitability Solution

Accurately weight about 25 mg of SPT2455M1 standard (Benzgalantamine standard) and pipette 5 mL of 1% standard solution into a 25-mL volumetric flask and dilute to volume with sample solvent and mix well.

IV. LOQ (0.05% of (−)-Benzgalantamine with Respect to Sample Solution)

Pipette 1 mL of standard solution into a 20-mL volumetric flask and dilute to volume with sample solvent and mix well. Further pipette 1 mL of this solution into a 100-mL volumetric flask and dilute to volume with sample solvent and mix well.

V. Sample Solution (0.5 mg/mL of Synthesized Benzgalantamine Gluconate, in Duplicate)

Accurately weigh about 12.5 mg of the respective sample to be determined into a 25-mL volumetric flask and dilute to volume with sample solvent and mix well.

### VI. Injection Sequence

Make one injection of the blank solution, one injection of sensitivity solution, one injection of system suitability solution, three injections of standard solution and one injection for each sample preparation. Insert one injection of standard solution after six sample injections at the end of the run.

### Results:

### I. Blank Evaluation

There were no interference peaks at the retention time of (±)-Benzgalantamine isomers.

### II. System Suitability Solution

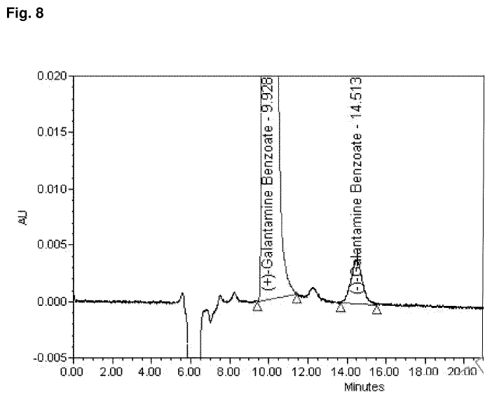

The resolution between (+)-Benzgalantamine and (−)-Benzgalantamine in the chromatogram of system suitability solution is 5.9. The representative chromatogram is shown in FIG. 8.

### III. Quantitation Limit (QL)

The signal-to-noise ratio (S/N) for 0.05% (−)-Benzgalantamine peak in the LOQ solution is 12.5 meeting S/N≥10.

### IV. Analysis of Benzgalantamine Samples

Three batches of synthesized benzgalantamine gluconate (Batch 71668AA004, 71714AA002 and 71714AA003) were selected for determination of the level of residual (−)-Benzgalantamine.

(−)-Benzgalantamine gluconate was not detected (ND; Table 13). By the method of the present invention thus advantageously Benzgalantamine can be produced in an enantiomeric pure form (i.e., (+)-Benzgalantamine).

TABLE 13
Chiral Purity of (+)-Benzgalantamine gluconate.

| | Quantity (%) | | |
|---|---|---|---|
| | Batch | Batch | Batch |
| Enantiomer | 711668AA004 | 71714AA002 | 71714AA003 |
| (+)-Benzgalantamine | 100 | 100 | 100 |
| gluconate | | | |
| (−)-Benzgalantamine | ND | ND | ND |
| gluconate | | | |

### 7. Polymorphism

The potential for benzgalantamine gluconate to exhibit polymorphism or different solid forms, such as crystal forms, was studied. It was determined that Form A anhydrate is the only anhydrous form, that can form hydrates, i.e., Form B tetrahydrate, Form C hemihydrate, and Form D dihydrate, in the presence of water, e.g. high humidity conditions.

Form A anhydrate is the stable anhydrous form from 0% RH to an undetermined value less than 0.12 aw (12% RH), but was shown to be kinetically stable above 12% RH; Form A exhibits significant hygroscopicity above 75% RH; quickly hydrating to Form D dihydrate when exposed to 75% RH (and eventually to Form B tetrahydrate with prolonged exposure) or, based on Dynamic Vapor Sorption (DVS) experiments, immediately to Form B tetrahydrate at and above 85% RH.

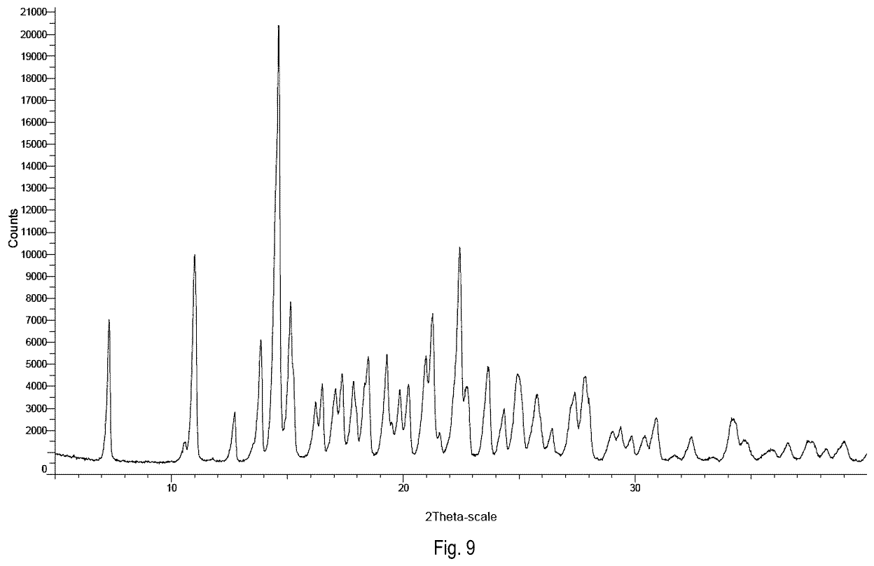

The X-ray powder diffraction (XRPD) pattern of Form A anhydrate (batch 71668AA001) is provided in FIG. 9.

Different ratios of MEK/H₂O in step 2 (overall MEK/H₂O ratio (vol./vol.; at steps 2.1 to 2.4 in Table 2) have been studied. The results indicated that the anhydrate Form A of Benzgalantamine gluconate can be obtained at an overall MEK/H₂O ratio from 10/1 to 15/1. The water content of the final Benzgalantamine (Form A) varied from 0.41% to 0.98% after the second drying step (step 2.13 in Table 2). Accordingly, the final Benzgalantamine should not comprise more than 1.2% in order to provide Form A.

Since the salt formation of Benzgalantamine is performed in a biphasic solvent system (MEK/H₂O), wherein the crystallization occurs in the interface of MEK/H₂O, it is further recommended controlling the water content at no more than 4% and ensure sufficient stirring during precipitation to provide Benzgalantamine in Form A.

### 8. Melting Behavior

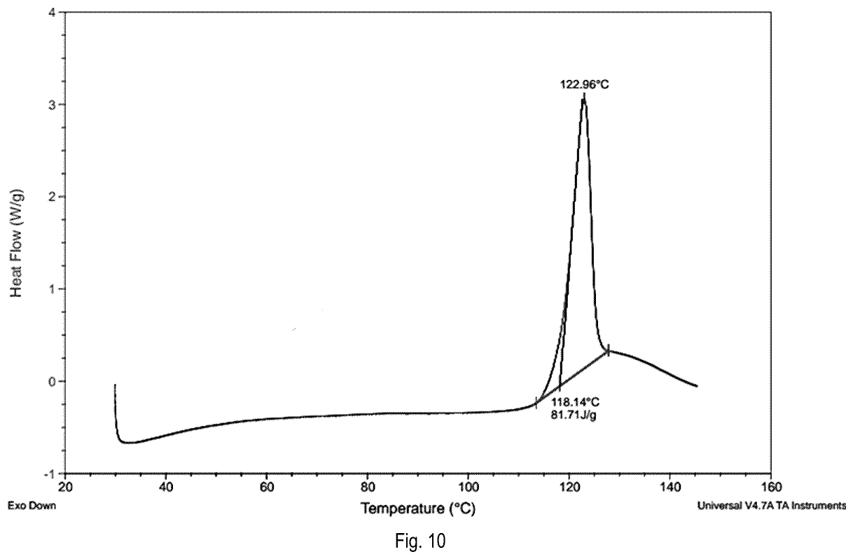

The melting behavior of several batches of Benzgalantamine gluconate was determined using differential scanning calorimetry (DSC) from 30° C. to 150° C. and a 10° C./min rate of increase. The results for 10 batches are provided in Table 14 and a typical DSC thermogram is provided in FIG. 10.

The onset of peak, defined as the melting point, ranged from 107.81° C. to 120.02° C. and decomposed from 112.52° C. to 124.11° C. indicated by unsymmetrical broad peak; When melting point was measured by USP<741> Class A Apparatus II, melting started at 110.8° C. to 111.5° C. followed by the sample color gradually changed to yellow without melting completely between 118.0° C. to 119.2° C., indicating that the sample decomposed.

TABLE 14
Differential Scanning Calorimetry (DSC),
Galantamine benzonate gluconate.

Thermal transition (° C.)

| Batch | Onset | Peak |
|---|---|---|
| 71668AA001 | 118.96 | 123.35 |
| 71668AA002 | 120.02 | 124.11 |
| 71668AA003 | 113.39 | 119.51 |
| 71668AA004 | 117.76 | 122.36 |
| 71668AA005 | 117.57 | 122.20 |
| 71668AA006 | 118.14 | 133.96 |
| 71714AA001 | 107.81 | 115.52 |
| 71714AA002 | 112.88 | 118.54 |
| 71714AA003 | 114.78 | 120.13 |
| 71714AA004 | 113.52 | 119.75 |

### 9. Hygroscopicity

The hygroscopicity of three batches benzgalantamine gluconate was evaluated at 25° C./80% RH for 24 h; the results are provided in Table 15. Based on this data, benzgalantamine gluconate is considered hygroscopic/slightly hygroscopic.

TABLE 15
Hygroscopicity of Benzgalantamine gluconate.

| Batch | Weight Gain (%) |
|---|---|
| 71668AA001 | 2.03 |
| 71668AA002 | 1.75 |
| 71668AA003 | 1.89 |

### III. Impurity Control:

The impurities can be categorized into two types based on their origin, where i) material impurities are those being carried over from starting material or generated due to trace amount of impurity from raw materials, and ii) process related impurities that are being generated via the synthetic process or the present invention or degradation.

The identification of impurities is conducted by screening of starting material, reaction mixture of each step, intermediates and final product by LC-MS, followed by proposing the possible structure based on the results.

### 1. Material Impurities

There are five specified impurities in Galantamine hydrobromide (SPT2455SM1)—Epi-Galantamine, Lycoramine, 0-desmethyl-galantamine, N-oxide of Galantamine and N-desmethyl-galantamine—which are derivatives or analogues of Galantamine and would also undergo esterification with benzoyl chloride in Step 1 to generate the corresponding impurities (Table 16). Further, there are several corresponding impurities that should be controlled as unspecified impurity at not more than 0.1% in Benzgalantamine gluconate.

TABLE 16
Corresponding impurities generated in step 1.

| | Origin from SPT2455SM1's | Identification in API |
|---|---|---|
| Corresponding Impurity | Specified Impurity | (criterion) |
| 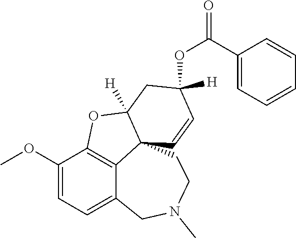 [SMILES: COc1ccc2c3c1O[C@H]1C[C@H](OC(=O)c4ccccc4)C=C[C@@]31CCN(C)C2] | Epi-Galantamine | Epi-Galantamine benzoylester (NMT 0.2%) |
| C₂₄H₂₅NO₄ | | |
| Exact Mass: 391.18 | | |
| Mol. Wt.: 391.46 | | |
| 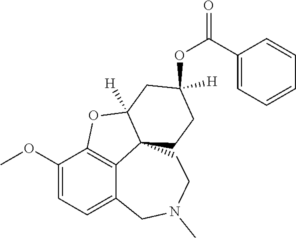 [SMILES: COc1ccc2c3c1O[C@H]1C[C@@H](OC(=O)c4ccccc4)CC[C@@]31CCN(C)C2] | Lycoramine | Unspecified impurity (NMT 0.1%) |
| C₂₄H₂₇NO₄ | | |
| Exact Mass: 393.19 | | |
| Mol. Wt.: 393.48 | | |
| 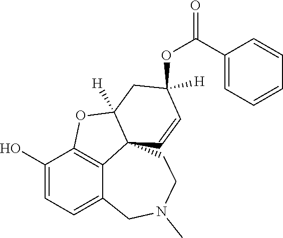 [SMILES: CN1CC[C@@]23C=C[C@H](OC(=O)c4ccccc4)C[C@@H]2Oc2c(O)ccc(c23)C1] | O-desmethyl-galantamine | Unspecified impurity (NMT 0.1%) |
| C₂₃H₂₃NO₄ | | |
| Exact Mass: 377.16 | | |
| Mol. Wt.: 377.43 | | |
| 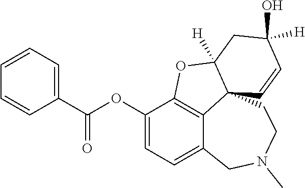 [SMILES: CN1CC[C@@]23C=C[C@H](O)C[C@@H]2Oc2c(OC(=O)c4ccccc4)ccc(c23)C1] | O-desmethyl-galantamine | Unspecified impurity (NMT 0.1%) |
| C₂₃H₂₃NO₄ | | |
| Exact Mass: 377.16 | | |
| Mol. Wt.: 377.43 | | |
| 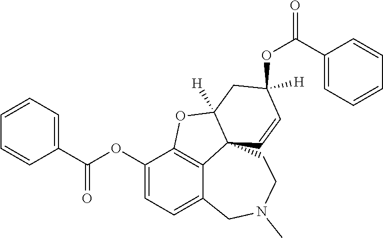 [SMILES: CN1CC[C@@]23C=C[C@H](OC(=O)c4ccccc4)C[C@@H]2Oc2c(OC(=O)c4ccccc4)ccc(c23)C1] | O-desmethyl-galantamine | Unspecified impurity (NMT 0.1%) |
| C₃₀H₂₇NO₆ | | |
| Exact Mass: 481.19 | | |
| Mol. Wt.: 481.54 | | |
| 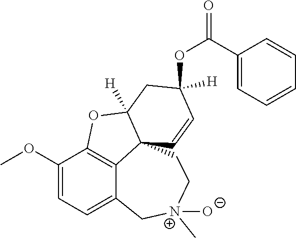 | N-oxide of galantamine | N-oxide Galantamine benzoylester (NMT 0.2%) |
| C₂₄H₂₅NO₅ | | |
| Exact Mass: 407.17 | | |
| Mol. Wt.: 407.46 | | |
| 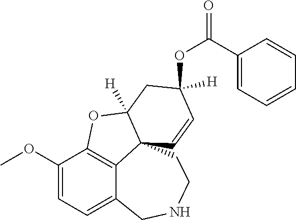 [SMILES: COc1ccc2c3c1O[C@H]1C[C@@H](OC(=O)c4ccccc4)C=C[C@@]31CCNC2] | N-desmethyl-galantamine | Nor-Galantamine benzoylester (NMT 0.6%) |
| C₂₃H₂₃NO₄ | | |
| Exact Mass: 377.16 | | |
| Mol. Wt.: 377.43 | | |
| 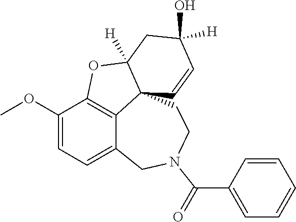 [SMILES: COc1ccc2c3c1O[C@H]1C[C@@H](O)C=C[C@@]31CCN(C(=O)c1ccccc1)C2] | N-desmethyl-galantamine | Unspecified impurity (NMT 0.1%) |
| C₂₃H₂₃NO₄ | | |
| Exact Mass: 377.16 | | |
| Mol. Wt.: 377.43 | | |
| 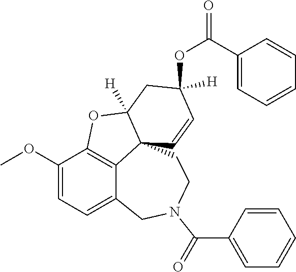 [SMILES: COc1ccc2c3c1O[C@H]1C[C@@H](OC(=O)c4ccccc4)C=C[C@@]31CCN(C(=O)c1ccccc1)C2] | N-desmethyl-galantamine | Unspecified impurity (NMT 0.1%) |
| C₃₀H₂₇NO₅ | | |
| Exact Mass: 481.19 | | |
| Mol. Wt.: 481.54 | | |

NMT: not more than; RT: retention time; RRT: relative retention time; SPT2455SM1 (Galantamine hydrobromide);
API: active pharmaceutical ingredient, i.e. Benzgalantamine gluconate.

### 2. Process Related Impurities

Four process related impurities during the production of Benzgalantamine gluconate are considered as potential impurities due to their growth of more than 0.10% during reaction. These impurities have been identified by LC-MS analysis and NMR (Table 17).

Further, one additional impurity MW886 (RT 1.82) is observed, when SPT2455SM1 (Galantamine hydrobromide) comprises higher amounts of epi-Galantamine. This impurity is considered to be the stereoisomer of process-related impurity MW887 (RT 1.84).

TABLE 17
Main process related impurities during the production of Benzgalantamine gluconate.

| | RRT in Testing | |
|---|---|---|
| Process Related Impurity | Method # | Description |
| 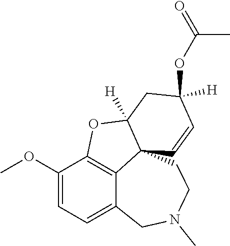 [SMILES: COc1ccc2c3c1O[C@H]1C[C@@H](OC(C)=O)C=C[C@@]31CCN(C)C2] | RRT ~0.48 in M2455001 | 1. This impurity is observed in Step 1. 2. During work-up, the transesterification of residual SPT2455SM1 with IPAc would occur in the stage of extraction with NaHCO₃ aqueous solution. 3. The highest level of this impurity in historical batches is 0.16%. |
| C₂₀H₂₇NO₃ | | |
| Exact Mass: 329.20 | | |
| Mol. Wt.: 329.43 | | |
| 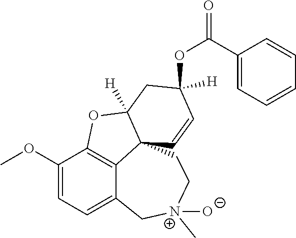 | RRT ~1.06 in M2455001 | 1. This impurity is due to the oxidation of SPT2455M1 by oxygen and observed in Step 1 & 2. 2. The formation of this impurity could be well controlled under N₂ atmosphere during reaction. |
| C₂₄H₂₅NO₅ | | |
| Exact Mass: 407.17 | | |
| Mol. Wt.: 407.46 | | |
| 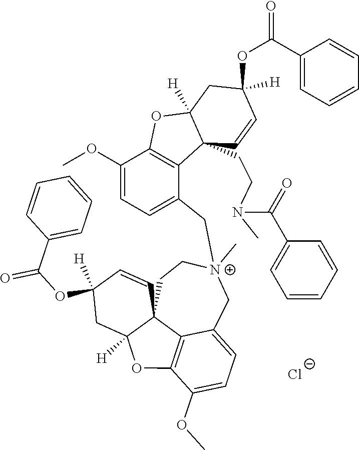 | RRT ~1.84 in M2455001 | 1. This impurity is observed in Step 1. 2. It is generated by the over- reaction of SPT2455M1 with residual BzCl. 3. It could be well controlled by the slow addition of BzCl in IPAc into SPT2455SM1 solution at low temperature. 4. The highest level of this impurity in historical batches is 2.53%. |
| C₅₅H₅₅N₂O₉⁺ | | |
| Exact Mass: 887.39 | | |
| Mol. Wt.: 888.03 | | |
| 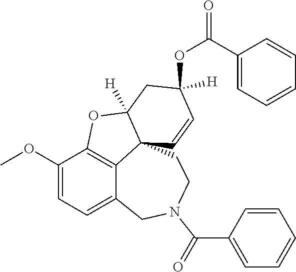 [SMILES: COc1ccc2c3c1O[C@H]1C[C@@H](OC(=O)c4ccccc4)C=C[C@@]31CCN(C(=O)c1ccccc1)C2] | RRT ~1.91 in M2455001 | 1. This impurity is observed in Step 1. 2. It is generated by the over- reaction of SPT2455M1 with residual BzCl. 3. It could be well controlled by the slow addition of BzCl in IPAc into SPT2455SM1 solution at low temperature. 4. The highest level of this impurity in historical batches is 0.67%. |
| C₃₀H₂₇NO₅ | | |
| Exact Mass: 481.19 | | |
| Mol. Wt.: 481.54 | | |

NMT: not more than; RT: retention time; RRT: relative retention time; SPT2455SM1 (Galantamine hydrobromide):
M2455001: HPLC method for determination of purity; IPAc: Isopropyl acetate: historical batches: batches previously
prepared; BzCl: Benzyl chloride.

### 3. Impurity Purge Rate Study (Material Impurities)

In order to elucidate the fate of the impurities during reaction, impurity-spiked experiments were conducted, wherein five specified impurities in Galantamine hydrobromide were studied, respectively. In this impurity-spiking experiment, there are three checking points in Step 1, including the reaction mixture (in-process control), organic layer after extraction, and isolated product, to monitor the amount of target impurity, giving its purge rate. If needed, the concerned impurity is further studied in the purge rate in Step 2.

### Epi-Galantamine

0.4 wt % of Epi-galantamine was mixed with Galantamine hydrobromide (SPT2455SM1) to perform Step 1.

The corresponding impurity—Epi-galantamine benzoylester—was observed at 0.46% in the reaction mixture. During work-up, this impurity could not be removed in the stage of extraction, but it was not detected by HPLC in the isolated Benzgalantamine (Table 18). This result indicated that criterion of Epi-galantamine in Galantamine hydrobromide set at not more than 0.15% is acceptable for the production of Benzgalantamine gluconate.

In the following tables B # refers to the respective batch number.

TABLE 18
Impurity-spiked study with Epi-Galantamine.

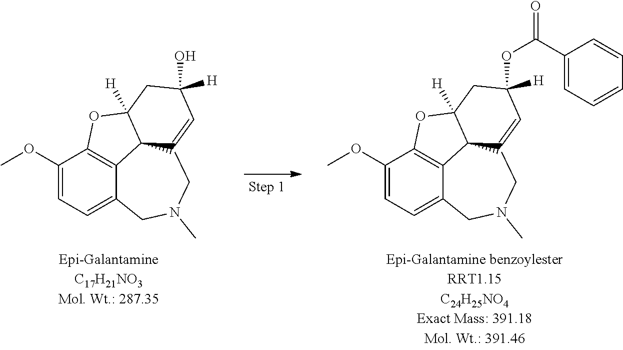 [SMILES: COc1ccc2c3c1O[C@H]1C[C@H](O)C=C[C@@]31CCN(C)C2 ; COc1ccc2c3c1O[C@H]1C[C@H](OC(=O)c4ccccc4)C=C[C@@]31CCN(C)C2]

| | RRT | |
|---|---|---|
| | | MW |
| | SPT2455M1 | 391 |
| B#2455_RD- | 1.00 | 1.15 |
| 00058-002 | 99.54 | 0.46 |
| (IPC, Reaction mixture) | | |
| 00058-003 | 99.55 | 0.45 |
| (Org. layer after extraction) | | |
| Purge rate (%) | | ~0 |
| (in the stage of extraction) | | |
| 00058-004 | 100 | ND |
| (Isolated M1) | | |
| Purge rate (%) | | 100 |
| (In the stage of crystallization) | | |

Note:
Only Benzgalantamine (SP2455M1 also termed M1) and target impurity were integrated.

### Lycoramine

0.2 wt % of Lycoramine was mixed with Galantamine hydrobromide (SPT2455SM1) to perform Step 1.

The corresponding impurity—MW393 (RRT 1.03) was observed at 0.20% in the reaction mixture. Afterwork-up, the isolated Benzgalantamine (SPT2455M1) still contained 0.20% of MW393 (RRT 1.03). Therefore, this impurity could not be removed in Step 1 (Table 19). Furthermore, this batch of Benzgalantamine (batch 2455_RD-00054-004) containing 0.20% of MW393 (RRT 1.09) was employed to perform salt formation in Step 2. It indicated that MW393 (RRT 1.03) was carried over to final API (Table 20). Based on these results, the criterion of Lycoramine in Galantamine hydrobromide should be tightened to not more than 0.08%.

TABLE 19
Impurity spiked study with Lycoramine.

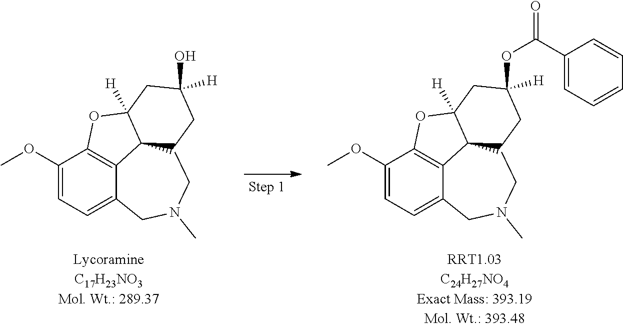 [SMILES: COc1ccc2c3c1O[C@H]1C[C@@H](O)CC[C@@]31CCN(C)C2 ; COc1ccc2c3c1O[C@H]1C[C@@H](OC(=O)c4ccccc4)CC[C@@]31CCN(C)C2]

| | RRT | |
|---|---|---|
| | | MW |
| | SPT2455M1 | 393 |
| B#2455_RD- | 1.00 | 1.03 |
| 00054-002 | 99.80 | 0.20 |
| (IPC, Reaction mixture) | | |
| 00054-003 | 99.80 | 0.20 |
| (Org. layer after extraction) | | |
| Pure rate (%) | | 0 |
| (in the stage of extraction) | | |
| 00054-004 | 99.80 | 0.20 |
| (Isolated M1) | | |
| Pure rate (%) | | 0 |
| (In the stage of crystallization) | | |

Note:
Only Benzgalantamine (SP2455M1 also termed M1) and target impurity were integrated.

TABLE 20
Examination of purge rate of MW393 (RTT 1.03) in step 2

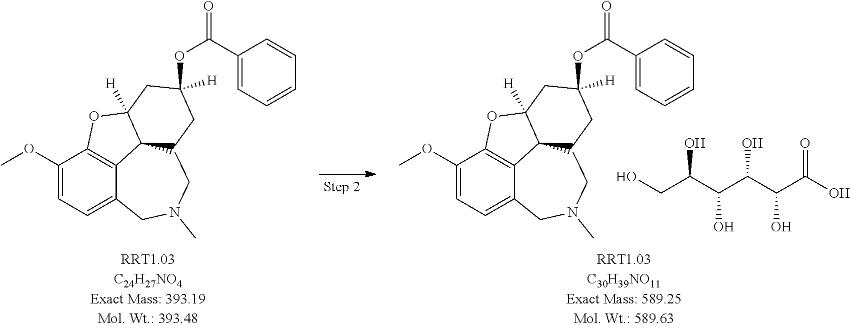

| | RRT | |
|---|---|---|
| | | MW |
| | SPT2455M1/API | 589 |
| B#2455_RD- | 1.00 | 1.03 |
| 0054-004 | 99.80 | 0.20 |
| (Isolated M1) | | |
| 00056-001 | 99.80 | 0.20 |
| (Isolated API) | | |
| Purge rate (%) | | 0 |
| (Step 2, salt formation) | | |

Note:
Only Benzgalantamine (SP2455M1 also termed M1) and target impurity were integrated.

O-desmethyl-galantamine

0.2 wt % of O-desmethyl-galantamine was mixed with Benzgalantamine (SPT2455SM1) to perform Step 1 (Table 21).

Three corresponding impurities—MW377 (RRT 0.78 & RRT 0.99) and MW481 (RRT 1.39) were observed at 0.07%, 0.01% and 0.21%, respectively, in the reaction mixture. After extraction, the purge rate for MW377 (RRT 0.78) was 43% and MW377 (RRT 0.99) was not detected by HPLC. MW481 (RRT 1.39) could not be removed in this stage of extraction. After isolation, the residual target impurities all could be removed completely. These results indicated that criterion of O-desmethyl-galantamine in Galantamine hydrobromide set at not more than 0.15% is acceptable for the production of Benzgalantamine gluconate.

TABLE 21
Impurity spiked study with O-desmethyl-galantamine.

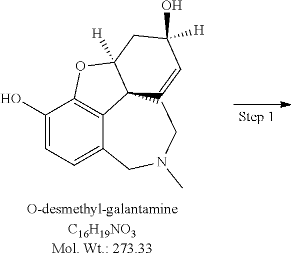 [SMILES: CN1CC[C@@]23C=C[C@H](O)C[C@@H]2Oc2c(O)ccc(c23)C1]

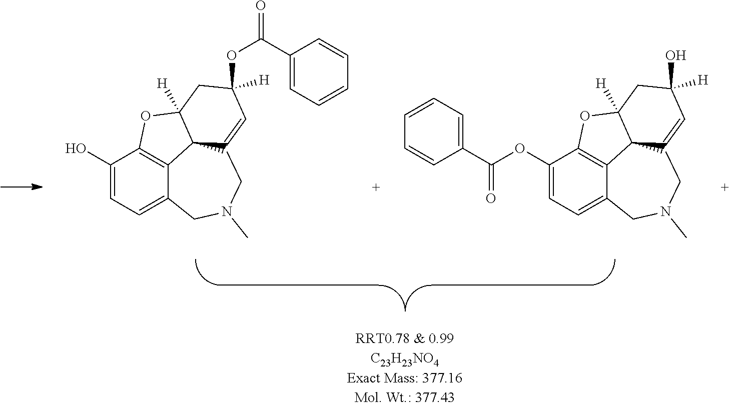 [SMILES: CN1CC[C@@]23C=C[C@H](OC(=O)c4ccccc4)C[C@@H]2Oc2c(O)ccc(c23)C1 ; CN1CC[C@@]23C=C[C@H](O)C[C@@H]2Oc2c(OC(=O)c4ccccc4)ccc(c23)C1]

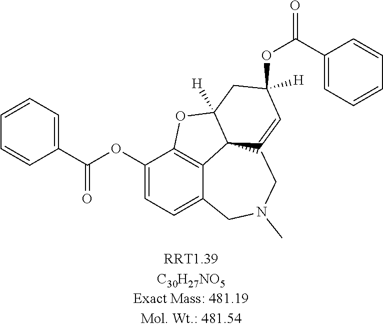 [SMILES: CN1CC[C@@]23C=C[C@H](OC(=O)c4ccccc4)C[C@@H]2Oc2c(OC(=O)c4ccccc4)ccc(c23)C1]

| | RRT | | | |
|---|---|---|---|---|
| | MW | MW | | MW |
| | 377 | 377 | SPT2455M1 | 481 |
| B#2455_RD- | 0.78 | 0.99 | 1.00 | 1.39 |
| 00046-003 | 0.07 | 0.01 | 99.72 | 0.21 |
| (IPC, Reaction mixture) | | | | |
| 00046-004 | 0.04 | ND | 99.77 | 0.19 |
| (Org. layer after extraction) | | | | |
| Purge rate (%) | 43 | 100 | | ~0 |
| (in the stage of extraction) | | | | |
| 00046-005 | ND | ND | 100 | ND |
| (Isolated M1) | | | | |
| Purge rate (%) | 100 | | | 100 |
| (In the stage of crystallization) | | | | |

Note:
Only Benzgalantamine (SP2455M1 also termed M1) and target impurity were integrated.

### N-oxide of Galantamine

0.2 wt % of N-oxide of galantamine was mixed with Galantamine hydrobromide (SPT2455SM1) to perform Step 1 (Table 22).

The corresponding impurity N-oxide of galantamine benzoylester was observed at 0.22% in reaction mixture. During work-up, the purge rate of this impurity was 64% in the stage of extraction. After isolation, only 0.02% of N-oxide galantamine benzoylester remained in Benzgalantamine (SPT2455M1). Therefore, the results indicated that criterion of N-oxide of galantamine in SPT2455SM1 set at not more than 0.20% is acceptable for the production of Benzgalantamine gluconate.

TABLE 22
Impurity-spiked study with N-oxide of Galantamine.

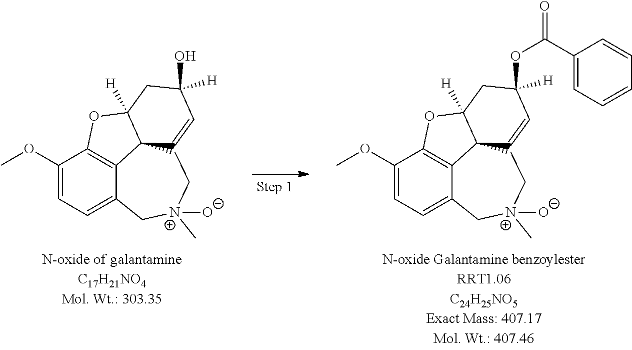

| | RRT | |
|---|---|---|
| | | MW |
| | SPT2455M1 | 407 |
| B#2455_RD- | 1.00 | 1.06 |
| 00055-002 | 99.78 | 0.22 |
| (IPC, Reaction mixture) | | |
| 00055-003 | 99.92 | 0.08 |
| (Org. layer after extraction) | | |
| Purge rate (%) | | 64 |
| (in the stage of extraction) | | |
| 00055-004 | 99.98 | 0.02 |
| (Isolated M1) | | |
| Purge rate (%) | | 75 |
| (In the stage of crystallization) | | |

Note:
Only Benzgalantamine (SP2455M1 also termed M1) and target impurity were integrated.

N-desmethyl-galantamine

0.2 wt % of N-desmethyl-galantamine was mixed with Galantamine hydrobromide (SPT2455SM1) to perform Step 1 (Table 23).

Only di-substituted impurity—MW481 (RRT 1.91)—was observed at 0.38% in reaction mixture. During work-up, this impurity could not be removed in the stage of extraction. After isolation, half the amount of MW481 (RRT 1.91) was purged out. This impurity also serves as process related impurity and could be removed completely in Step 2. Therefore, the criterion of N-desmethyl-galantamine in SPT2455SM1 set at not more than 0.2% is acceptable for the production of Benzgalantamine gluconate.

TABLE 23
Impurity-spiked study with N-desmethyl-galantamine.

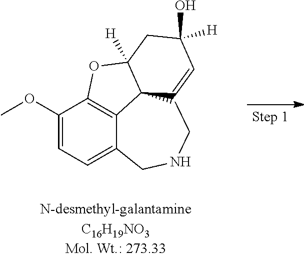 [SMILES: COc1ccc2c3c1O[C@H]1C[C@@H](O)C=C[C@@]31CCNC2]

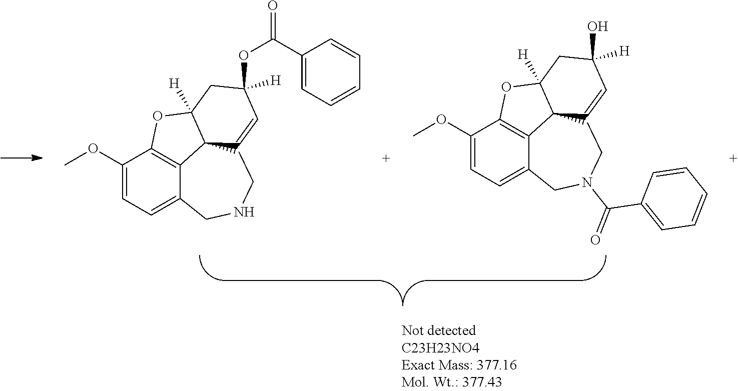 [SMILES: COc1ccc2c3c1O[C@H]1C[C@@H](OC(=O)c4ccccc4)C=C[C@@]31CCNC2 ; COc1ccc2c3c1O[C@H]1C[C@@H](O)C=C[C@@]31CCN(C(=O)c1ccccc1)C2]

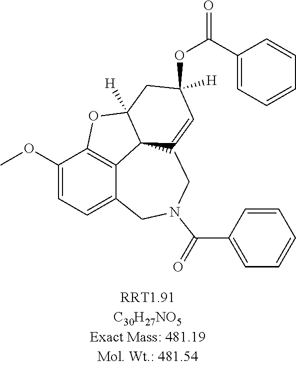 [SMILES: COc1ccc2c3c1O[C@H]1C[C@@H](OC(=O)c4ccccc4)C=C[C@@]31CCN(C(=O)c1ccccc1)C2]

| | RRT | |
|---|---|---|
| | | MW |
| | SPT2455M1 | 481 |
| B#2455_RD- | 1.00 | 1.91 |
| 00050-002 | 99.62 | 0.38 |
| (IPC, Reaction mixture) | | |
| 00050-003 | 99.62 | 0.38 |
| (Org. layer after extraction) | | |
| Purge rate (%) | | 0 |
| (in the stage of extraction) | | |
| 00050-004 | 99.81 | 0.19 |
| (Isolated M1) | | |
| Purge rate (%) | | 50 |
| (In the stage of crystallization) | | |

Note:
Only Benzgalantamine (SP2455M1 also termed M1) and target impurity were integrated.

### 4. Impurity Purge Rate Study (Process Related Impurities)

As listed in Table 17, four impurities would be generated during the production of Benzgalantamine gluconate and have been identified by LC-MS and NMR. Since the quantity of these impurities in Step 1 would increase to more than 0.10%, they should be seen as process-related impurities. Therefore, the purge rate study in the downstream step (Step 2) was conducted.

### MW329 (RRT 0.49) Impurity

This impurity is generated by the transesterification of Galantamine hydrobromide (SPT2455SM1) with IPAc in Step 1 (Scheme 3). Usually, it is removed completely after isolation of Benzgalantamine (SPT2455M1). Considering the risk of carry-over to next step, the impurity-spiked study in Step 2 was conducted mixing 0.5 wt % of MW 329 (RRT 0.48) with SPT2455M1. The result indicated that 100% purge rate was obtained.

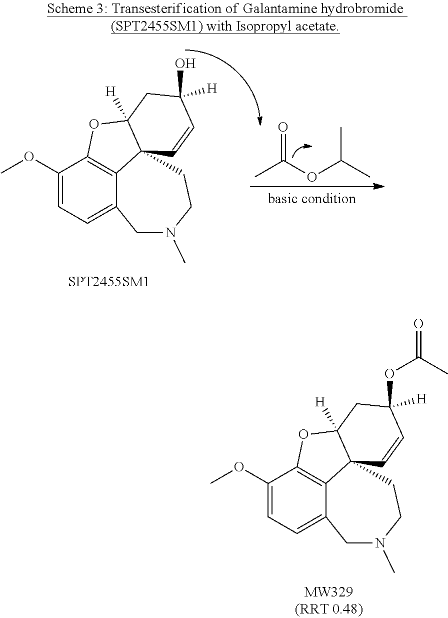

MW407 (RRT 1.06) Impurity (N-oxide galantamine benzoylester)

This impurity is generated by the oxidation of Benzgalantamine (SPT2455M1) with oxygen during production of Benzgalantamine gluconate. As mentioned in above (material related impurities), this impurity exhibits a good purge rate in Step 1. The purge rate in Step 2 was also studied, but only 20% of N-oxide galantamine benzoylester could be removed. Therefore, the overall process should preferably be performed under nitrogen atmosphere to control the amount of this impurity.

### MW887 (RRT 1.84) and MW481 (RRT 1.91) Impurities:

These two impurities are generated during the addition of benzyl chloride (BzCl), wherein the Benzgalantamine (SPT2455M1) would react with the residual BzCl to undergo the substitution at N atom (Scheme 4). The reaction performed at low temperature reduces the formation of these two impurities. Further, Benzgalantamine (SPT2455M1) containing MW887 at 2.53% and MW481 at 0.67% was employed to study the purge rate in Step 2. The results indicated these levels of impurity can be purged out (Table 24).

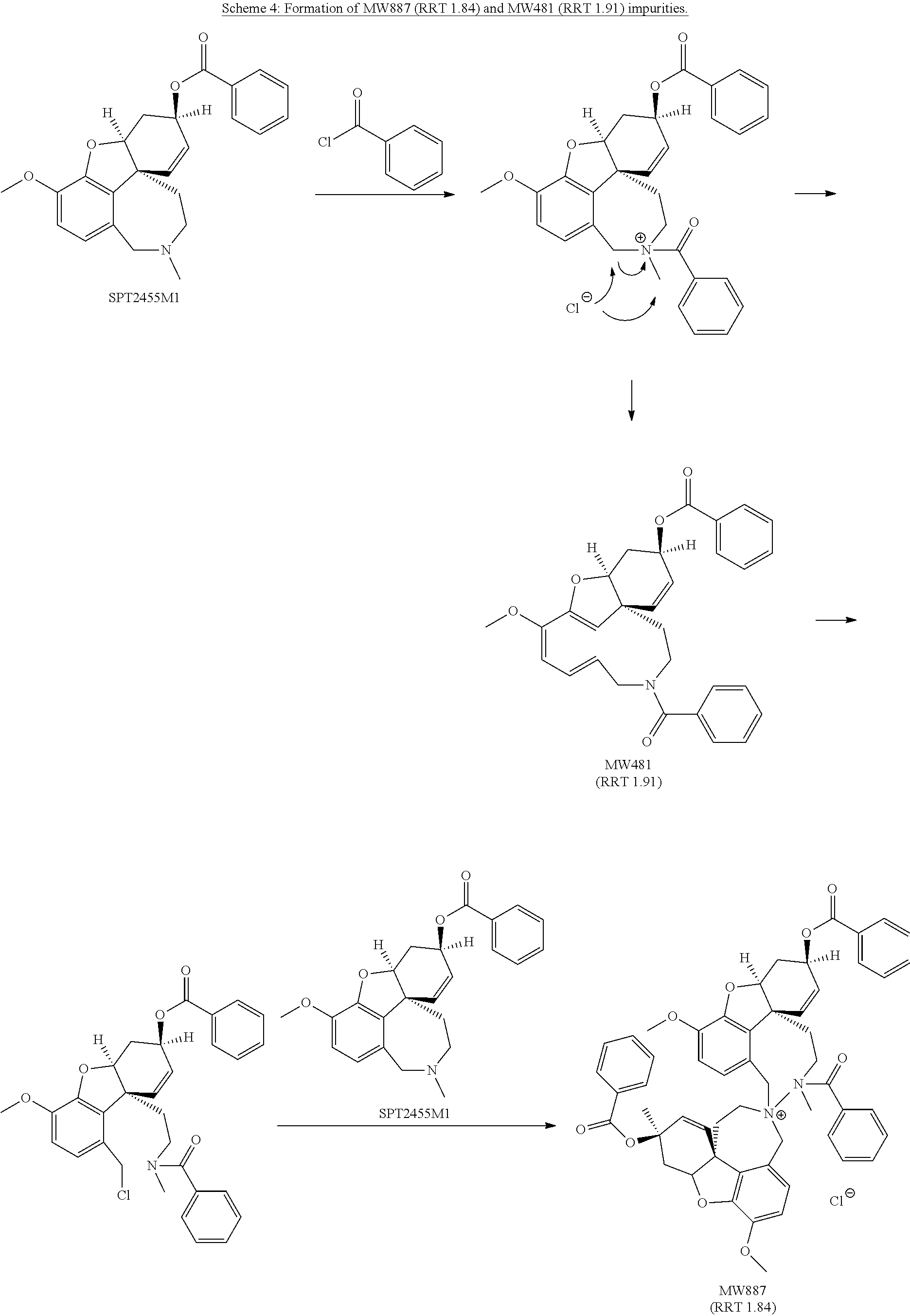

TABLE 24
Purge rate study of MW887 and MW481 (RRT 1.91) impurities.

| | RRT | | | | | | |
|---|---|---|---|---|---|---|---|
| | | | | | | MW | MW |
| | SM1 | BzOH | M1/API | N-oxide | | 887 | 481 |
| B#2455_RD- | 0.18 | 0.60 | 1.00 | 1.06 | 1.82 | 1.84 | 1.91 |
| 00005-021 (M1) | 0.15 | 0.08 | 99.41 | 0.02 | 0.14 | 2.53 | 0.67 |
| 00007-009 (API) | ND | ND | 99.88 | 0.03 | ND | 0.09 | ND |
| Purge rate (%) (Step 2) | | | | | | 96 | 100 |

### MW886 (RT. 1.82) Impurity

This purity was observed in batches in which SPT2455SM1 (Galantamine hydrobromide) comprising a higher amount of epi-Galantamine within the acceptance criteria was used (0.18% epi-Galantamine). This impurity was 886 Da as indicated by LC-MS and is considered to be the stereoisomer of the MW 887 (RT 1.84) impurity.

The level of RRT1.82 was low (0.01% ~0.02%) in step 1 and not detected in step 2.

### 5. Specification of Galantamine Hydrobromide

Based on these results, the specification of SPT2455SM1 (Galantamine hydrobromide) when used for synthesis of Benzgalantamine gluconate should be according to Table 25.

TABLE 25
Specification of Galantamine hydrobromide.

| Method # | Testing Items | Specification |
|---|---|---|
| M0000030 | Appearance | White to off- |
| | | white powder |
| M0000002 | Identification (FT-IR) | Conform to standard |
| M1113009 | Identification (HPLC) | Conform to standard |
| M1113010 | Identification (Bromide) | Conforms |
| M1113003 | Assay (HPLC) | NLT 98.0% and |
| | (dried basis) | NMT 102.0% |
| | Enantiomeric Purity (HPLC) | |
| M1113006 | (+)-Galantamine | NMT 0.10% |
| | Purity (HPLC) | |
| M1113001 | Epi-Galantamine | NMT 0.20% |
| | Lycoramine | NMT 0.08% |
| | O-desmethyl-galantamine | NMT 0.15% |
| | N-oxide of galantamine | NMT 0.20% |
| | N-desmethyl-galantamine | NMT 0.2% |
| | Other max. unknown impurity | NMT 0.10% |
| | Total impurities | NMT 1.0% |
| | Residual Solvent | |
| | (GC Headspace) | |
| M1113002 | Methanol | NMT 500 ppm |
| | Ethanol | NMT 5,000 ppm |
| | Isopropanol | NMT 500 ppm |
| | Tetrahydrofuran | NMT 360 ppm |
| M0000036 | Loss on Drying | NMT 0.5% |
| M0000040 | Specific Rotation | NLT −99° and |
| | | NMT −92° |
| M0000007 | Residual on Ignition | NMT 0.1% |

### 6. Specification of Benzgalantamine Gluconate

The specification of Benzgalantamine gluconate produced according to the process of the present invention is according to Table 26.

TABLE 26
Specification of Benzgalantamine gluconate.

| Test | Specification | Result |
|---|---|---|
| Appearance | White to pale | Conforms |
| | yellow powder | |
| X-ray Powder Diffraction | Conforms to | Conforms |
| | anhydrate form | |
| Identification and | | |
| Gluconic Cid | | |
| Content: NMR | | |
| Identification: NMR (1H) | Conforms to reference | Conforms |
| | spectrum | |
| Gluconic acid | NLT 0.45 moles and | 0.50 moles |
| | NMT 0.55 moles | |
| Assay and Impurity: HPLC | | |
| Galantamine | NLT 98.0% and | 100.1% |
| Benzoate Gluconate | NMT 102.0% | |
| (anhydrous basis) | | |
| Galantamine hydrobromide | NMT 0.15% | N.D. |
| Nor-Galantamine Benzoate | NMT 0.15% | N.D. |
| Epi-Galantamine Benzoate | NMT 0.15% | N.D. |
| N-Oxide Galantamine | NMT 0.15% | N.D. |
| Benzoate | | |
| Any unspecified impurity | NMT 0.1% | LT 0.05% |
| Total impurities | NMT 1.0% | LT 0.05% |
| Residual Solvents: Headspace | | |
| Acetonitrile | NMT 410 ppm | N.D. |
| Isopropyl acetate | NMT 5,000 ppm | N.D. |
| Triethylamine | NMT 5,000 ppm | N.D. |
| n-Heptane | NMT 5,000 ppm | N.D. |
| Methyl ethyl ketone | NMT 5,000 ppm | N.D. |
| Residue on Ignition | NMT 0.2% | LT 0.05% |
| Specific Rotation | NLT +3.9 deg and | +5.0 deg |
| | NMT +6.8 deg | |
| Water Content: Karl Fischer | NMT 1.2% | 0.30% |
| Microbial Enumeration Tests: | | |
| Total Aerobic Microbial Count | NMT 100 CFU/g | LT 1 CFU/g |
| Total Combined Yeasts | NMT 100 CFU/g | LT 1 CFU/g |
| and Molds Count | | |
| Specified Microorganisms: | | |
| Escherichia coli | Absent | Conforms |
| Pseudomonas aeruginosa | Absent | Conforms |
| Staphylococcus aureus | Absent | Conforms |

NMT: not more than,
N.D.: not detected,
NLT: not less than,
LT: less than

### IV. Process Optimization and Large Scale Manufacture

Within the development of the synthesis process of section I., studies have been conducted to optimize different steps of the process, for example with regards to yield, purity and reaction time.

### 1. Optimization of Step 1

### a) Equivalent of Benzyl Chloride (Table 1—Steps 1.5 to 1.9)

The over-reaction, process related impurities (MW 887 & MW 481, see above in section III.) in Benzgalantamine (SPT2455M1) are generated because of an excess amount of benzyl chloride (BzCl) (see Table 27). Especially, higher amounts of BzCl lead to more formation of MW 887 impurity. Therefore, reducing equivalent of BzCl was examined to evaluate the impact on the reaction.

TABLE 27
Comparison of Over-reaction impurities

| | | Process impurities | |
|---|---|---|---|
| | | MW887 | MW481 |
| Step 1 Esterification (overall 1.2 eq. BzCl) | | (RRT 1.84) | (RRT 1.91) |
| Lab's Demo batch | 2455_RD-00064-009 | 0.02 | 0.14 |
| GMP batches | 71668AA001 | 0.72 | 0.10 |
| | 71668AA002 | 0.83 | 0.10 |
| | 71668AA003 | 0.84 | 0.09 |

While the amount of BzCl was reduced to 1.1 equiv., the reaction became slower than that condition in 1.2 equiv. of BzCl (Table 28). The results indicated that residual Galantamine hydrobromide (SPT2455SM1) (3.37%) was observed after 2 hours versus residual SPT2455SM1 (0.67%) using 1.2 equiv. of BzCl, resulting in lower yield. The reaction time would have to be extended to 20 hours at 1.1 equiv. BzCl, giving a comparable conversion of SPT2455SM1 as for 1.2 equiv. BzCl. Considering the production efficiency and the risk for moisture control, the amount of at least 1.2 equiv. is recommended.

It is concluded that the process impurity level of MW 887 could be controlled at a maximum of approx. 0.84% when the benzoyl chloride is kept at 1.2 equivalents. In the downstream process, these impurities MW 887 and MW 481 could be effectively purged into mother liquor with good removal rate and be controlled by any unspecified impurity at NMT 0.1% in the final specification of the synthesis product.

TABLE 28
Impurity profile of reaction mixture after addition of overall
1.1 equiv. of BzCl or 1.2 equiv. BzCl. SM1: SPT2455SM1
(Galantamine hydrobromide); M1: SPT2455M1 (Benzgalantamine).
The reaction was performed in 50 g scale of SM1.

| | SM1 | M1 | MW 887 | MW 481 |
|---|---|---|---|---|
| | 0.18 | 1.00 | 1.84 | 1.91 |
| 00068-002 | 3.37 | 96.58 | ND | 0.05 |
| (2 hrs) | | | | |
| 00068-003 | 3.02 | 96.94 | ND | 0.04 |
| (4 hrs) | | | | |
| 00068-004 | 2.69 | 97.26 | ND | 0.04 |
| (6 hrs) | | | | |
| 00068-005 | 2.47 | 97.49 | ND | 0.04 |
| (8 hrs) | | | | |
| 00068-006 | 2.35 | 97.61 | ND | 0.04 |
| (12 hrs) | | | | |
| 00068-007 | 0.77 | 99.17 | ND | 0.05 |
| (20 hrs) | | | | |
| 00011-002^{d} | 0.67 | 99.22 | 0.04 | 0.07 |
| (2 hrs) | | | | |

^{d}The reaction was performed in 30 g scale of SM1 using 1.2 equiv. of BzCl.

### 2. Demonstration Run

### Step 1—Preparation of Benzgalantamine

Following the modified process in Step 1, the preparation of SPT2455M1 was demonstrated in 100 g scale of SPT2455SM1 (Table 29). The results revealed that acceptable purity of SPT2455M1 was obtained with a 93% isolated yield.

TABLE 29
Impurity profile of Benzgalantamine (SPT2455SM1;
M1) after process optimization

| | Impurity profile | | | | |
|---|---|---|---|---|---|
| | | N-oxide of | | | |
| | | Galan- | | | |
| | M1 | tamine | MW887 | MW481 | |
| | (RRT | (RRT | (RRT | (RRT | Yield |
| Batch | 1.00) | 1.06) | 1.84) | 1.91) | (%) |
| 2455_RD- | 99.93 | 0.02 | 0.03 | 0.02 | 93 |
| 00079-005 | | | | | |
| (M1) | | | | | |

### Step 2—Preparation of Benzgalantamine Gluconate

The above Benzgalantamine (SPT2455M1) was used to demonstrate the preparation of Benzgalantamine gluconate following the optimized process. The 93% isolated yield of Benzgalantamine gluconate was obtained and the analytical results were summarized in Table 30.

TABLE 30
Analytical results for demonstration batch of Benzgalantamine gluconate.
Sample batch: 2455_RD-00080-008

| Testing | | Testing | | Assess- |
|---|---|---|---|---|
| method | Description | results | | ment |
| M0000030 | Appearance | White solid | | Pass |
| M2455013 | XRPD | Conforms to anhydrate form | | Pass |
| M2455003 | ID and Salt | Conforms to reference spectrum | | Pass |
| | Content | Gluconic acid | 0.50 moles | |
| | by NMR | | | |
| M2455001 | Assay and | Galantamine | 100.8% | Pass |
| | Impurity | Benzoate | | |
| | by HPLC | Gluconate | | |
| | | (anhydrous | | |
| | | basis) | | |
| | | Galantamine | N.D. | |
| | | hydrobromide | | |
| | | Nor-Galantamine | N.D. | |
| | | Benzoate | | |
| | | Epi-Galantamine | N.D. | |
| | | Benzoate | | |
| | | N-Oxide | 0.08% | |
| | | Galantamine | | |
| | | Benzoate | | |
| | | Individual | N.D. | |
| | | unknown | | |
| | | impurity | | |
| | | Total impurities | 0.08% | |
| M2455002 | Residual | ACN | N.D. | Pass |
| | Solvents | IPAc | N.D. | |
| | by GC | TEA | N.D. | |
| | | n-Heptane | N.D. | |
| | | MEK | N.D. | |

| | | | |
|---|---|---|---|
| M2455006 | pH | 5.1 | Pass |
| M0000026 | ROI | 0.01% | Pass |
| M2455008 | Specific | +5.2° | FIO |
| | Rotation | | |
| M2455009 | MP by DSC | 117.2° C. and 112.4° C. | FIO |
| | | (duplicate tests) | |
| M2455012 | Water by KF | 0.44% | FIO |

FIO: for information only;
MP: melting point;
ID: identity;
KF: Karl-Fisher titration;
ROI: residual on ignition

### 3. Large Scale Demonstration Run

An additional demonstration run has been performed producing 2.29 kg of Benzgalantamine gluconate from 1.8 kg Galantamine hydrobromide (batch 71731 DA001). The batch met the expected qualifications (IPCs according to table 3). The isolation yield was 90%. Further, a small particle size and narrow particle size distribution was observed (see Table 31). Thus, the produced Benzgalantamine gluconate may directly be processed as API without the need for an additional milling step.

TABLE 31
Particle size distribution of large scale batch produced
by the optimized process (batch 71731DA001).

| Batch 71731DA001 | D (v, 0.1) (μm) | D (v, 0.5) (μm) | D (v, 0.9) (μm) |
|---|---|---|---|
| Preparation #1 | 11.456 | 37.683 | 72.823 |
| Preparation #2 | 15.996 | 45.510 | 87.276 |
| Average | 13.726 | 41.597 | 80.050 |

### 4. Stability of Preparation

Benzgalantamine gluconate Form A was produced according to the method above. The specification of Benzgalantamine gluconate was maintained over 48 months in storage, without deviation from the specification provided above in Table 26 (Table 32). Benzgalantamine gluconate was stored in HOPE bottles/AL bag/Double LOPE bags at 25±2 deg C., 60±5% RH, for 48 months. Samples were assessed at 1, 2, 3, 6, 9,12,18, 24, 36, and 48 months storage. Multiple batches were tested with analogous results. Results upcoming for 60-month storage.

TABLE 32
Benzgalantamine gluconate after storage.

| Test | Specification | Result |
|---|---|---|
| Appearance | White to pale | White powder |
| | yellow powder | |
| X-ray Powder Diffraction | Conforms to | Conforms to |
| | anhydrate form | anhydrate form |
| Assay and Impurity: HPLC | | |
| Galantamine Benzoate Gluconate | NLT 98.0% and | 100.8% |
| (anhydrous basis) | NMT 102.0% | |
| Galantamine hydrobromide | NMT 0.15% | N.D. |
| Nor-Galantamine Benzoate | NMT 0.15% | N.D. |
| Epi-Galantamine Benzoate | NMT 0.15% | N.D. |
| N-Oxide Galantamine Benzoate | NMT 0.15% | LT 0.05% |
| Any unspecified impurity | NMT 0.1% | LT 0.05% |
| Total impurities | NMT 1.0% | LT 0.05% |
| Water Content: Karl Fischer | NMT 1.2% | 0.25% |
| Microbial Enumeration Tests: | | |
| Total Aerobic Microbial Count | NMT 100 CFU/g | LT 1 CFU/g |
| Total Combined Yeasts | NMT 100 CFU/g | LT 1 CFU/g |
| and Molds Count | | |

NMT: not more than,
N.D.: not detected,
NLT: not less than,
LT: less than

1. A process for preparing benzgalantamine 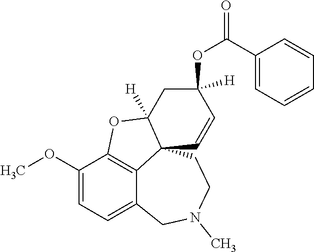 [SMILES: COc1ccc2c3c1O[C@H]1C[C@@H](OC(=O)c4ccccc4)C=C[C@@]31CCN(C)C2]
comprising addition of a benzoyl halide to a galantamine salt.

2. The process according to claim 1, wherein the addition takes place in an organic solvent in the presence of a catalyst and a base.

3. The process according to claim 2, wherein the catalyst is a nucleophilic catalyst.

4. The process according to claim 1, wherein the benzoyl halide is benzoyl chloride.

5. The process according to claim 1, wherein the galantamine salt is galantamine hydrobromide.

6. The process according to claim 1, wherein the benzoyl halide is added at ≤1.7 equivalents of the galantamine salt.

7. The process according to claim 1, wherein the benzoyl halide is added at ≤1.5 equivalents of the galantamine salt.

8. The process according to claim 1, wherein the benzoyl halide is added at 1.2±0.5 equivalents of the galantamine salt.

9. The process according to claim 1, wherein the addition of benzoyl halide to galantamine salt occurs at ≤10° C.

10. The process according to claim 1, wherein the addition of benzoyl halide to galantamine salt occurs at 0±5° C.

11. The process according to claim 3, wherein:
a. the organic solvent is acetonitrile (ACN),
b. the nucleophilic catalyst is 4-dimethylaminopyridine (DMAP),
c. the base is triethylamine (Et₃N), and/or
d. the benzoyl halide is added in isopropyl acetate (IPAc).

12. The process according to claim 4, comprising the addition of benzoyl chloride to galantamine hydrobromide in the presence of a nucleophilic catalyst and a base, wherein the benzoyl chloride is added at 1.15 to 1.7 equivalents of the galantamine hydrobromide.

13. The process according to claim 12, wherein the benzoyl chloride is added at 1.2±0.5 equivalents of the galantamine hydrobromide at −10 to 10° C.

14. The process according to claim 1, wherein the addition of a benzoyl halide to a galantamine salt to form a mixture occurs at a pre-determined temperature of ≤20° C., and said mixture is stirred for 60-180 minutes at said pre-determined temperature, and subsequently warmed to an ambient temperature and stirred for 3-18 hours, followed by optional washing and drying.

15. The process according to claim 1, comprising additionally the formation of a salt of benzgalantamine subsequent to the process of claim 1.

16. The process according to claim 15, comprising the addition of a gluconolactone to benzgalantamine in methyl ethyl ketone (MEK), to prepare a gluconate salt of benzgalantamine.

17. A composition comprising or consisting of a reaction product prepared by the process according to claim 1.

18. The composition according to claim 17, wherein the reaction product comprises one or more of the following impurities:

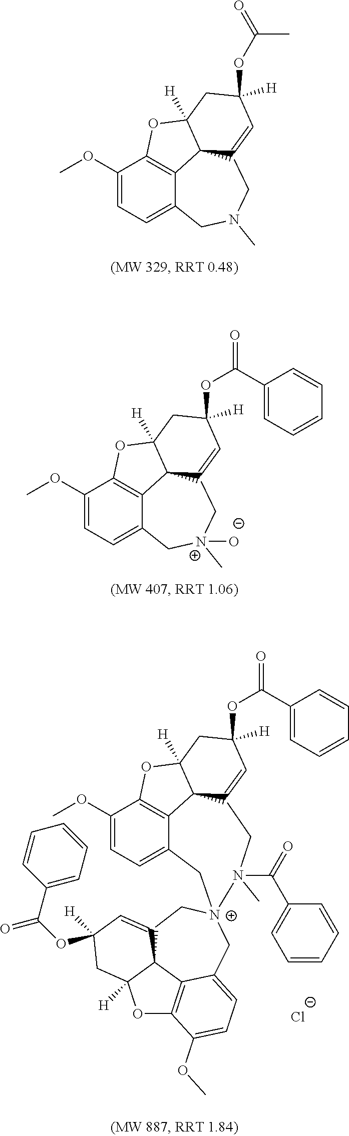

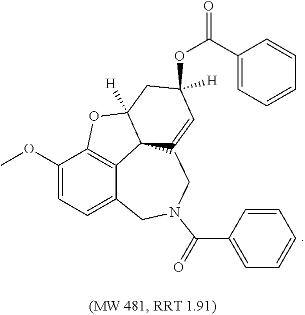 [SMILES: COc1ccc2c3c1O[C@H]1C[C@@H](OC(=O)c4ccccc4)C=C[C@@]31CCN(C(=O)c1ccccc1)C2]

19. The composition according to claim 18, wherein the reaction product comprises at least one of the following two impurities: 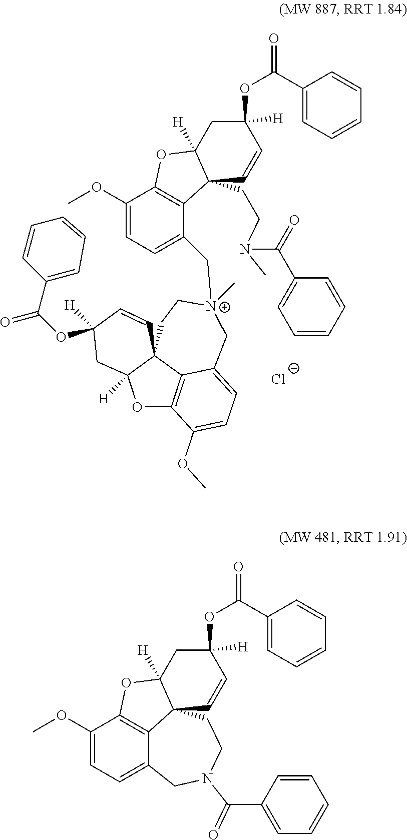
present at an amount of ≤3.5% when assessed by the area under the curve from an HPLC analysis of the reaction product.

20. A pharmaceutical composition comprising a composition according to claim 18 together with a pharmaceutically acceptable carrier.
